(19) United States
(12) Patent Application Publication
(10) Pub. No.: US 2016/0216259 A1
(43) Pub. Date: Jul. 28, 2016

(54) ISOTHERMAL TITRATION CALORIMETRY METHODS FOR EVALUATION OF THERMODYNAMIC BINDING PROPERTIES

(71) Applicant: The Penn State Research Foundation, University Park, PA (US)

(72) Inventors: Ji Woong Chang, State College, PA (US); Robert M. Rioux, State College, PA (US)

(21) Appl. No.: 15/007,947

(22) Filed: Jan. 27, 2016

Related U.S. Application Data
(60) Provisional application No. 62/108,381, filed on Jan. 27, 2015.

(51) Int. Cl. G01N 33/557; G01N 25/48

(52) U.S. Cl. CPC G01N 33/557; G01N 25/4866

(57) ABSTRACT

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the binding characteristic in real-time, producing a determined binding characteristic. Binding characteristics determined according to methods of the present invention include any one or more of ΔH, ΔS, ΔG, equilibrium binding constant (K), and binding stoichiometry (n).

### REFERENCE TO RELATED APPLICATION

This application claims priority from U.S. Provisional Patent Application Ser. No. 62/108,381, filed Jan. 27, 2015, the entire content of which is incorporated herein by reference.

### GRANT REFERENCE

This invention was made with government support under Grant No. DE-FG02-07ER46414, awarded by the Department of Energy. The Government has certain rights in the invention.

### FIELD OF THE INVENTION

The present invention relates generally to methods for evaluation of thermodynamic binding properties. According to specific aspects of the present invention, isothermal titration calorimetry methods for evaluation of thermodynamic binding properties are provided.

### BACKGROUND OF THE INVENTION

Isothermal titration calorimetry (ITC) is a powerful technique for understanding binding interactions between receptors and ligands in biology, material science and nanotechnology. It allows for the determination of thermodynamic binding parameters (free energy, enthalpy, and entropy) and binding stoichiometry in a single experiment by fitting the binding isotherm to a suitable binding model.

However, isothermal titration calorimeters and methods of their use are slow and limited and “real-time” methods for determination of thermodynamic binding properties are lacking.

### SUMMARY OF THE INVENTION

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the binding characteristic in real-time, producing a determined binding characteristic.

Binding characteristics determined according to methods of the present invention include any one or more of ΔH, ΔS, ΔG, equilibrium binding constant (K), and binding stoichiometry (n).

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the binding characteristic in real-time, wherein the total concentration of ligand injected is known, thereby producing a determined binding characteristic.

Isothermal titration calorimetry methods for determining one or more binding characteristics of a ligand and a receptor according to aspects of the present invention are described wherein the ligand and receptor are characterized by high binding affinity where K_{d} is lower than 1 nM.

Isothermal titration calorimetry methods for determining one or more binding characteristics of a ligand and a receptor according to aspects of the present invention are described wherein no additional heat flow values are obtained once the second derivative is determined to be equal to zero, thereby shortening the time to producing a determined binding characteristic compared to an incremental isothermal titration calorimetry method.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the binding characteristic in real-time, wherein the calculating is performed substantially as described herein, thereby producing a determined binding characteristic.

Isothermal titration calorimetry methods for determining one or more binding characteristics of a ligand and a receptor according to aspects of the present invention include continuous injection of the ligand using a syringe pump.

Computer programs for determining one or more binding characteristics of a ligand and a receptor using heat flow values obtained by an isothermal titration calorimetry method are provided according to aspects of the present invention wherein the computer program is operative to calculate the binding characteristic in real-time, producing a determined binding characteristic displayed to a user.

Computer programs for determining one or more binding characteristics of a ligand and a receptor using heat flow values obtained by an isothermal titration calorimetry method are provided according to aspects of the present invention wherein the computer program is operative to calculate the binding characteristic in real-time by incorporating a known total concentration of ligand injected into a sample cell of a calorimeter, producing a determined binding characteristic displayed to a user.

Computer programs for determining one or more binding characteristics of a ligand and a receptor using heat flow values obtained by an isothermal titration calorimetry method are provided according to aspects of the present invention wherein the computer program is operative to calculate the binding characteristic in real-time, producing a determined binding characteristic displayed to a user, and wherein the ligand and receptor are characterized by high binding affinity where K_{d} is lower than 1 nM.

Computer programs for determining one or more binding characteristics of a ligand and a receptor using heat flow values obtained by an isothermal titration calorimetry method are provided according to aspects of the present invention wherein the computer program is operative to calculate the binding characteristic in real-time by incorporating a known total concentration of ligand injected into a sample cell of a calorimeter, producing a determined binding characteristic displayed to a user, and wherein the ligand and receptor are characterized by high binding affinity where K_{d} is lower than 1 nM.

Computer programs for determining one or more binding characteristics of a ligand and a receptor using heat flow values obtained by an isothermal titration calorimetry method are provided according to aspects of the present invention wherein the computer program is operative to calculate the binding characteristic in real-time, and wherein the program provides a signal indicating that no additional heat flow values are obtained once the second derivative is determined to be equal to zero, producing a determined binding characteristic displayed to a user.

Computer programs for determining one or more binding characteristics of a ligand and a receptor using heat flow values obtained by an isothermal titration calorimetry method are provided according to aspects of the present invention wherein the computer program is operative to calculate the binding characteristic in real-time by incorporating measurement of the total concentration of ligand injected into a sample cell of a calorimeter, and wherein the program provides a signal indicating that no additional heat flow values are obtained once the second derivative is determined to be equal to zero, producing a determined binding characteristic displayed to a user.

Computer programs for determining one or more binding characteristics of a ligand and a receptor using heat flow values obtained by an isothermal titration calorimetry method are provided according to aspects of the present invention wherein the computer program is operative to calculate the binding characteristic substantially as described herein.

Isothermal titration calorimeter systems are provided according to aspects of the present invention wherein an isothermal titration calorimeter is in signal communication with a computer, the computer including a program for determining one or more binding characteristics of a ligand and a receptor using heat flow values obtained by an isothermal titration calorimetry method are provided according to aspects of the present invention wherein the computer program is operative to calculate the one or more binding characteristics in real-time, producing one or more determined binding characteristics displayed to a user.

Isothermal titration calorimeter systems are provided according to aspects of the present invention wherein an isothermal titration calorimeter in flow communication with a syringe pump for continuous injection of a ligand into a sample cell of the isothermal titration calorimeter, wherein the isothermal titration calorimeter is in signal communication with a computer, the computer including a program for determining one or more binding characteristics of a ligand and a receptor using heat flow values obtained by an isothermal titration calorimetry method are provided according to aspects of the present invention wherein the computer program is operative to calculate the one or more binding characteristics in real-time, producing one or more determined binding characteristics displayed to a user.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the one or more binding characteristics in real-time, producing one or more determined binding characteristics.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the one or more binding characteristics, wherein calculating the total concentration of ligand injected is known, producing one or more determined binding characteristics.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the one or more binding characteristics, wherein the total concentration of ligand injected is known, wherein the ligand and receptor are characterized by high binding affinity where K_{d} is lower than 1 nM, producing one or more determined binding characteristics.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the one or more binding characteristics, wherein the total concentration of ligand injected is known, wherein the ligand and receptor are characterized by high binding affinity where K_{d} is in the range of 10 mM-0.1 pM, producing one or more determined binding characteristics

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the one or more binding characteristics, wherein the total concentration of ligand injected is known, and wherein no additional heat flow values are obtained once the second derivative is determined to be equal to zero, thereby shortening the time to producing one or more determined binding characteristics compared to an incremental isothermal titration calorimetry method, producing one or more determined binding characteristics.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the one or more binding characteristics, wherein the total concentration of ligand injected is known, wherein the ligand and receptor are characterized by high binding affinity where K_{d} is lower than 1 nM, and wherein no additional heat flow values are obtained once the second derivative is determined to be equal to zero, thereby shortening the time to producing one or more determined binding characteristics compared to an incremental isothermal titration calorimetry method, producing one or more determined binding characteristics.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the one or more binding characteristics, wherein the total concentration of ligand injected is known, wherein the ligand and receptor are characterized by high binding affinity where K_{d} is in the range of 10 mM—0.1 pM, and wherein no additional heat flow values are obtained once the second derivative is determined to be equal to zero, thereby shortening the time to producing one or more determined binding characteristics compared to an incremental isothermal titration calorimetry method, producing one or more determined binding characteristics.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the one or more binding characteristics in real-time, wherein the calculating is performed substantially as described herein, producing one or more determined binding characteristics.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous, wherein a syringe pump is used for continuously injecting the ligand; obtaining heat flow values indicative of binding of the ligand to the receptor; and calculating the one or more binding characteristics in real-time, producing one or more determined binding characteristics.

Isothermal titration calorimetry methods for determining one or more binding characteristics of a ligand and a receptor according to aspects of the present invention include injecting a ligand into a sample cell of a calorimeter, the sample cell containing a receptor, wherein the injecting is continuous; obtaining heat flow values indicative of binding of the ligand to the receptor; and calculating the one or more binding characteristics, wherein calculating the calculating is in real-time and wherein no additional heat flow values are obtained once the second derivative is determined to be equal to zero, thereby shortening the time to producing the one or more determined binding characteristics compared to an incremental isothermal titration calorimetry method.

Isothermal titration calorimetry methods for determining one or more binding characteristics of a ligand and a receptor according to aspects of the present invention include injecting a ligand into a sample cell of a calorimeter, the sample cell containing a receptor, wherein the ligand and receptor are characterized by high binding affinity where K_{d} is lower than 1 nM, wherein the injecting is continuous; obtaining heat flow values indicative of binding of the ligand to the receptor; and calculating the one or more binding characteristics, wherein calculating the calculating is in real-time and wherein no additional heat flow values are obtained once the second derivative is determined to be equal to zero, thereby shortening the time to producing one or more determined binding characteristics compared to an incremental isothermal titration calorimetry method.

### BRIEF DESCRIPTION OF THE DRAWINGS

FIG. 1A is a graph illustrating results obtained by incremental ITC methodology;

FIG. 1B is a graph illustrating results obtained by methods according to aspects of the present invention in which [L]_{T} is used for real-time evaluation of binding characteristics;

FIG. 2A is a schematic representation of competitive binding;

FIG. 2B is a schematic representation of binding at two independent binding sites;

FIG. 3A is a schematic of a method according to aspects of the present invention for real-time evaluation used to determine the thermodynamic binding parameters for single independent site binding from the binding isotherm with respect to [L]_{T} and its derivative;

FIG. 3B is a schematic of a method according to aspects of the present invention for obtaining the values at the inflection point and deconvolution of the total heat of binding to the individual heat of binding and dissociation due to displacement of the low-affinity ligand;

FIG. 3C is a schematic of a method according to aspects of the present invention which demonstrates how data at inflection points and the individual heats of binding associated with each binding site for a system consisting of two independent binding sites is obtained;

FIG. 4A is a graph representing the binding isotherms for binding of Ba²⁺ to EDTA at 25° C. by incremental titration, along with the integrated heats and the corresponding best fit utilizing the single independent binding site model;

FIG. 4B is a graph representing the raw data for the binding of Ba²⁺ to EDTA at 25° C. using the continuous injection method and the corresponding best fit to the binding isotherm with respect to [L]_{T};

FIG. 5A is a graph showing experimental data and fit results in a competitive binding site model obtained with incremental injections;

FIG. 5B is a graph showing experimental data and fit results in a competitive binding site model obtained with continuous injection;

FIG. 6A is a graph showing experimental data and fit results in a two independent binding site model obtained with incremental injections;

FIG. 6B is a graph showing experimental data and fit results in a two independent binding site model obtained with continuous injection;

FIG. 7A is a graph showing experimental results using the differential form and difference of cumulative heat by using a finite difference;

FIG. 7B is a graph showing simulation results using the differential form and difference of cumulative heat by using a finite difference;

FIG. 8 is a graph showing a step input of 0.2 ptcal/s electric pulse and response of the ITC;

FIG. 9A is a graph showing the raw data of binding isotherm (top) for binding of Ca²⁺ to EDTA at 25° C. through a continuous injection method using a continuous injection method according to aspects of the present invention and binding isotherm with respect to [L]_{T} and the corresponding best fit (bottom);

FIG. 9B is a graph showing the raw data of binding isotherm (top) for binding of desthiobiotin to avidin at 25° C. through a continuous injection method using a continuous injection method according to aspects of the present invention and binding isotherm with respect to [L]_{T} and the corresponding best fit (bottom).

FIG. 10A is a graph showing single independent binding for lipoic acid using a continuous injection method according to aspects of the present invention;

FIG. 10B is a graph showing competitive binding for desthiobiotin using a continuous injection method according to aspects of the present invention;

FIG. 11 is a graph showing an example of the evaluation of binding constant for high affinity system (K_{d}=nano-molar, pico-molar, and femto-molar) depending on the injection rate and acquisition rate;

FIG. 12 is graphical interpretation of three real roots for a cubic equation;

FIG. 13 is a flow diagram illustrating isothermal titration calorimetry methods for determining one or more binding characteristics of a ligand and a receptor according to aspects of the present invention; and

FIG. 14 is a schematic diagram of an isothermal titration calorimeter in signal communication with a computer, the computer having a program relating to isothermal titration calorimetry methods for determining one or more binding characteristics of a ligand and a receptor according to aspects of the present invention.

### DETAILED DESCRIPTION OF THE INVENTION

The singular terms “a,” “an,” and “the” are not intended to be limiting and include plural referents unless explicitly stated otherwise or the context clearly indicates otherwise.

Broadly described, a calorimeter used in isothermal titration calorimetry includes two cells, a reference cell and a sample cell. A sensor of the calorimeter detects thermal differences between the two cells and measures heat that is either absorbed or released due to the interaction of a “ligand” and a “receptor” and allows determination of the binding affinity, stoichiometry, and entropy and enthalpy of the binding reaction in solution.

The term “ligand” as used herein refers to the binding partner injected into the calorimeter and the term “receptor” refers to the binding partner present in the sample cell in the calorimeter contacted by the injected “ligand.” The terms “ligand” and “receptor” are used to refer to binding partners of various types and include, but are not limited to, the ligand/receptor interaction as the terms are typically used in biological systems. Thus, binding partners encompassed by the terms ligand and receptor as used in the context of isothermal titration calorimetry systems and methods includes, but is not limited to, antigen/antibody; antigen/antigen binding antibody fragment; hormone/receptor; lectin/carbohydrate; enzyme/enzyme substrate; ligand/receptor; ion/chelator; and other such binding partners which specifically interact.

Previous methods based on an incremental injection approach fit the integrated heats of each successive injection to an ITC equation developed for the chosen binding model in order to evaluate the thermodynamic binding parameters; equilibrium binding constant (K), binding stoichiometry (n), and enthalpy of binding (AH). One of the main limitations of performing ITC experiments through the incremental injection method is the resulting small number of integrated heat data points (the data points are limited by the number of injections). This is particularly detrimental when one has to determine the thermodynamics of binding for systems with high affinity ligand-receptor systems. In general, a dissociation constant, K_{d} (or 1/K) is limited to the range 1 nM<K_{d}<10 mM, and evaluated thermodynamic parameters for a ligand-receptor system with a high binding affinity are less reliable because of similar step function shape of binding isotherms. As a result, the acquisition errors around the step propagate the error of the evaluated parameters.

Incremental injection methods are limited to the equilibrium constants range of 10⁴-10⁹ M⁻¹ (or dissociation constant, K_{d} range of 10 mM—1 nM). In a high affinity system, characterized by a K_{d} is lower than 1 nM, using an incremental injection mode, the binding isotherm shows two plateaus and no data or only a single data point between the plateaus of the binding isotherm. As binding affinity increases, the number of data points near the inflection point is very low. Therefore, the thermodynamic properties evaluated with the incremental injection method are not accurate and are unreliable below K_{d}=1 nM. Methods of the present invention extend the dynamic range of isothermal calorimeters and allow determination of one or more binding characteristics of a ligand and a receptor in a single binding site model or a two binding site model where K_{d} is in the range of 10 mM—0.1 pM.

Previous techniques for determining equilibrium constants for high affinity ligand-receptor binding have relied on 1) a tag and/or surface modification of either the ligand or receptor or 2) addition of a low affinity ligand for competitively binding to the receptor, even though the added ligand can alter binding environment of high-affinity ligand and receptor.

The methods according to aspects of the present invention include continuous injection of the ligand and provide an exact solution for binding isotherms expressed in terms of the total concentration of injecting ligand ([L]), rather than as a function of the concentration of free ligand ([L]). The present invention enables a reduced number of calculations by use of only one calculation per data point due to an exact solution of ITC equations while previous technique requires iteration for calculation of the heat, i.e. several calculations per data point.

As an example, the evaluation of thermodynamic binding parameters through an equation for the binding isotherm using [L](unmeasurable variable) always requires more than one iteration for each data point, such as, in this example 10 iterations for each data point, where the time taken for the injection of ligand is 1 h, with data acquired every second, the iteration method requires 360000 iterations (=3600 data points×10 iterations with given thermodynamic binding parameters×10 iterations to change thermodynamic binding parameters for the best fit). By contrast, the solution using [L]_{T} (measurable variable) in the present invention does not require iterations for each data point since each data point is solved by an exact solution of ITC calculation and needs only the necessary iterations (10 in this example) to evaluate the thermodynamic binding parameters to acquire the best fit to the experimental data (dQ/dt).

For example, a sequence of calculation included in methods according to aspects of the present invention includes: Step 1. Comparing experimental data and calculated data with guessed values of K, ΔH, and n. (guessed value is given by users); Step 2. If error is greater than a user acceptable tolerance value, then change K, ΔH, and n; Step 3. repeat “Step 1 and 2” until error is at or lesser than the user acceptable tolerance value; Step 4. End.

In this sequence, iteration requires only for adjusting K, ΔH, and n.

By contrast, a sequence of calculation for the previous method includes: Step 1. compare guessed K and calculated K using [L] of the first data point. The initial [L] is used as [L]_{T}; Step 2. If error is greater than a user acceptable tolerance value, then change [L]; Step 3. repeat “Steps 1 and 2” until error is at or lesser than the user acceptable tolerance value; Steps 4-6. if the first data point is calculated, then calculate [L] of the second data point by repeating “Steps 1-3”; Step 7. repeat “Steps 1-3” for every remaining data points (if data point is 3600 (=1 hour experiment and a data point is measured every second), then the number of steps is now 3×3600=10800). Step 10801. Compare experimental data and calculated data with guessed K, ΔH, and n. (guessed value is given by users); Step10802. If error is greater than a user acceptable tolerance value, then change K, ΔH, and n; Step 10803. repeat “Steps 10801 and 10802” until error is at or lesser than the user acceptable tolerance value; Step10804. repeat “Steps 1-10803” until error is at or lesser than the user acceptable tolerance value.

This type of method requires an iteration for each data point. After calculating each data point, an additional iteration is required to determine K, ΔH, and n. Therefore, the number of iterations becomes very large.

As used herein, the term “user acceptable tolerance” is a value that a user defines according to aspects of methods of the present invention. Tolerance is a criteria for convergence of an iterative calculation. If the error is less than the tolerance with a definite number of iterations, then the value is converged.

If error is larger than the tolerance with an indefinite number of iterations, then the value is diverged (has not converged). Error can be calculated as absolute value of (calculated value with previous iteration—calculated value with current iteration)/calculated value with previous iteration. A typical number defined for tolerance is 0.001, although lower or higher numbers can be used according to a user's preference.

Methods according to aspects of the present invention provide determination of binding characteristics of ligand-receptor binding in real-time.

Methods according to aspects of the present invention provide determination of binding characteristics of high affinity ligand-receptor binding directly by a continuous injection method using label-free and surface modification-free methodology.

Methods according to aspects of the present invention provide determination of nanomolar and picomolar dissociation constants in high affinity ligand/receptor binding systems.

Described herein are concentrations of ligand and receptor, and flow rates, for obtaining precise equilibrium constants. The results are shown in Tables 4-6.

Determination of binding characteristics of ligand/receptor binding systems, including high affinity binding systems, has utility in various applications, such as but not limited to, drug design. In addition to determination of an equilibrium constant K, measurement of enthalpy of binding (ΔH) and other binding characteristics plays an important role in discovery and designing of new drugs because ΔH is related to drug pharmacokinetics.

Binding characteristics determined according to methods of the present invention include any one or more of ΔH, ΔS, ΔG, equilibrium binding constant (K), and binding stoichiometry (n).

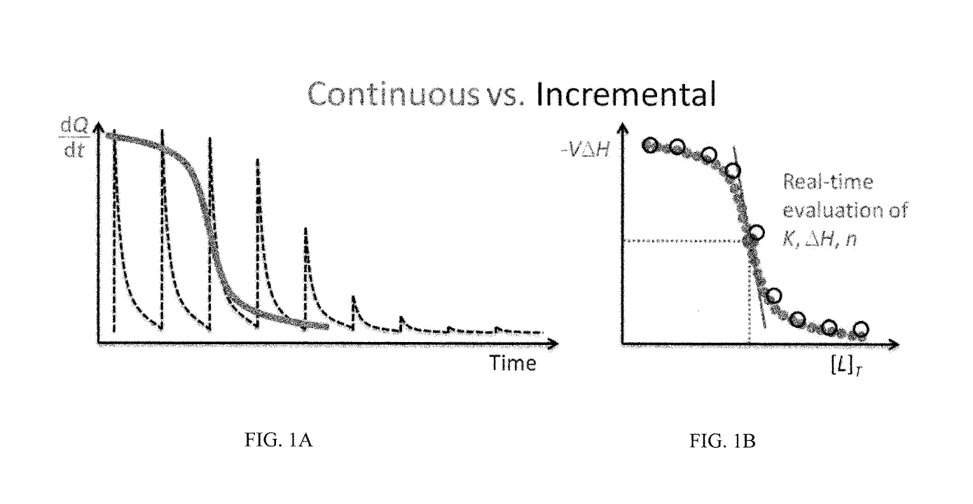

FIGS. 1A and 1B are graphs illustrating differences between results obtained by prior incremental ITC methodology (FIG. 1A) compared to results achieved by methods according to aspects of the present invention in which [L]^{r} is used for real-time evaluation of binding characteristics (FIG. 1B).

In addition to reduced experimental and analysis time, values at inflection point(s) of the binding isotherm as a function of [L]_{T} allow a user to obtain thermodynamic binding parameters in real time during the titration in the continuous injection method (i.e., a complete set of data is required with the incremental injection method). The raw heat flow (dQ/dt) data is easily converted to heat with respect [L]_{T}, (dQ/d[L]_{T}), by dividing dQ/dt by d[L]_{T}/dt.

According to aspects of the present invention, methods of ITC provide the solutions for the competitive binding site model and two independent binding sites model as a function of [L]_{T}, for 1:1 binding stoichiometries and for stoichiometries other than 1:1.

Provided by aspects of the present invention is an ITC method incorporating an equation for the binding isotherm for the competitive binding model and the two independent binding sites model with respect to the total concentration of the injecting ligand. These ITC equations for the binding isotherm enable the analysis of the binding isotherms with the continuous injection method which reduces the experimental time because the required equilibration time between injections is eliminated. Continuous injection method necessitates the differential form of ITC equations for the binding isotherms rather than incremental injection method. The confidence interval of binding constants with 99% confidence level are reduced compared to the incremental injection method for single independent binding site, competitive binding site, and two independent binding sites due to the large number of data points acquired during the continuous injection method. Therefore, analyses using the developed ITC equations in a differential form for the binding isotherms with continuous injection method are faster and more precise simultaneously to obtain the thermodynamic binding parameters than analyses using cumulative heats with finite differences by incremental injection method.

Methods according to aspects of the present invention provide determination and evaluation of thermodynamic binding parameters in real-time during a continuous injection configuration. The values at inflection point(s) enable a user to obtain thermodynamic binding properties of the receptor-ligand binding by solving algebraic equations in continuous injection method because the corresponding values from the incremental injection method vary depending on the number of injections. Methods according to aspects of the invention are applicable to assessment of binding partners having a single independent binding site, competitive binding sites, and two independent binding sites to estimate the accuracy of a real-time evaluation method by solving the appropriate system of algebraic equations. Real-time evaluation method rather than fitting after an experiment completes is useful to obtain the thermodynamic binding parameters with high precision during the experiment.

Methods according to aspects of the present invention are implemented by a user controlled isothermal titration calorimeter including a continuous injection apparatus.

Determination of one or more binding characteristics of a ligand and receptor in an isothermal titration calorimeter having at least one reference cell and at least one sample cell includes continuous injection of a ligand into the sample cell by an injection apparatus. The sample cell contains the receptor. A temperature modulator is included in the calorimeter to maintain the same temperature in the reference and sample cells. When the temperature of the sample cell changes due to binding of the ligand and receptor, energy is expended by the temperature modulator to match the temperature of the reference cell and sample cells. The energy expended is proportional to the change in temperature in the sample cell and a raw heat flow value is obtained. A plurality of heat flow values is obtained over time while the ligand is continuously injected into the sample cell at a predetermined rate. One or more binding characteristics of the ligand and receptor is calculated using the obtained heat flow values.

Aspects of methods according to the present invention are implemented by an apparatus for calculating the one or more binding characteristics of the ligand and receptor using the obtained heat flow values. The apparatus may be integral to the calorimeter or may be a separate apparatus to which obtained heat flow values generated by the calorimeter are transferred for processing. The apparatus is optionally dedicated to performing operations according to methods described herein. Alternatively, the apparatus may be a multipurpose computer configured to perform operations according to methods described herein by a computer program stored in the computer and/or encoded in a computer readable medium implemented by the computer.

The obtained heat flow values generated by the calorimeter may be transferred to a computer by any of various methods such as storage in computer memory such as a chip, disk, hard drive, flash drive, memory drive, optical storage drive, and the like, and/or transmission of the obtained heat flow values via any transmission medium such as but not limited to wires, cables or optical fibers, for transmission of signals such as but not limited to electrical, optical, acoustic, digital or infrared to a computer memory.

A computer processing unit accesses the obtained heat flow values in the computer memory and performs calculations described herein to determine one or more binding characteristics of the ligand and receptor and displays the resulting determined binding characteristic of the ligand and receptor.

A computer program is provided according to aspects of the present invention for determining at least one binding characteristic of a ligand and receptor. The computer program runs on a computer, accessing obtained heat flow values and calculating the resulting determined binding characteristics of the ligand and receptor.

Methods of calculating the binding characteristics of the ligand and receptor are described hereinbelow.

Mathematical Models for Binding

The number of binding sites per receptor is unity for a number of binding systems involving ions, small molecules, and biomolecules. However, the total number of binding sites, a part of receptor molecule which binds one ligand molecule, can be different from the total number of receptor such as nanoparticles and biomolecules with polyvalent interaction. For instance, when a receptor contains more than one binding site, the total number of binding sites, S_{T}, is greater than the total number of receptors, M_{T}, ([S]_{T}>[M]_{T}.). In contrast, the total number of binding sites (S_{T}) can be less than the total number of receptors (M_{T}) ([S]_{T}<[M]_{T}). When characterizing the interaction between receptor, M, and ligand, L, the binding equilibrium can be described by quantifying [S] and [L]. Due to one ligand occupying one binding site, i.e., S+L [symbol] SL:

$$K = \frac{[ SL ]}{[ S ] [ L ]} \qquad ( 1 )$$

where, K and [SL] are the binding equilibrium constant and the concentration of bound ligand, respectively.

Using the concentration of binding sites instead of receptors, i.e.,

[S]=[M]/n (2)

where n is the binding stoichiometry between M and L. This is a generalized description and expands the utilization of the developed ITC equations for the binding isotherm to any system that displays non-unity binding. Current commercial software supports change of the stoichiometry to any non-unity value for the single independent binding site and the two independent binding sites models only.

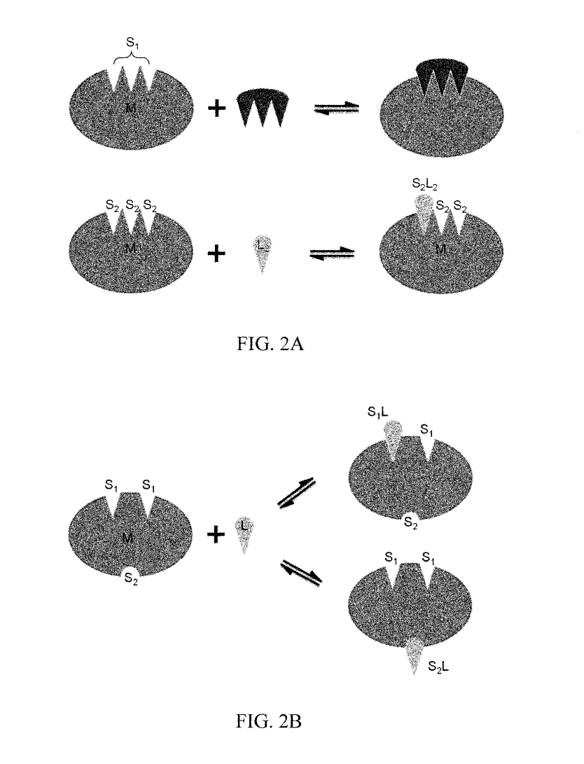

FIGS. 2A and 2B are schematic representations of (2A) competitive binding and (2B) two independent binding sites model, where M=receptor, L₁=Ligand 1 for competitive binding, L₂=Ligand 2 for competitive binding, S₁=binding site 1, S₂=binding site 2, S₁L₁=L₁ bound with S₁, and S₂L₂=bound L₂ with S₂, L=ligand for two independent binding sites, S₁L=L bound with S₁, and S₂L=L bound with S₂.

FIGS. 2A and 2B represent different binding equilibria between receptors and ligands. The receptor, M, has three binding subunits in FIG. 2A. A ligand, L₁, can bind with M using all three binding subunits; therefore, the three subunits become one binding site for L₁, i.e., S₁. The other ligand, L₂, can bind with Musing one subunit, therefore, each subunit becomes one binding site for L₂, i.e., S₂. The relationship between receptor and ligand is [M]_{T}=[S₁]_{T}, 3×[M]_{T}=[S₂]T. Thus, the stoichiometry for L₁ and L₂ are 1 and ⅓, respectively. The receptor in FIG. 2B consists of two types of subunits which can potentially be occupied by the same ligand. One ligand binds with each subunit, therefore, this can lead to two different combinations between the receptor and the ligand; 2×[M]_{T}=[S₁]_{T}, [M]_{T}=[S₂]_{T}. Thus, the stoichiometry for two independent binding sites are ½ and 1, respectively.

Single Independent Binding Site Model

In the single independent binding site model, the receptor may have several binding sites but each site is thermodynamically identical and has the same thermodynamic affinity for the ligand. Here, the ITC equation for the binding isotherm for the single independent model is re-formulated using the definition for stoichiometry as described in FIGS. 2A and 2B. The total conservation of M and L is shown below.

[M]_{T}=[M]+n[SL]

[L]_{T}=[L]+[SL] (3)

where [M]_{T} and [L]_{T} are the total concentration of receptor and ligand, respectively. Using eqns. (1-3), a quadratic equation for [SL] is obtained as follows.

Kn[SL]²−(K[M]_{T}+n+K[L]_{T}n)[SL]+K[L]_{T}[M]_{T}=0 (4)

The solution of eqn. (4) yields two real roots out of which only one root provides a physically meaningful value for [SL].

$$[ SL ] = \frac{\begin{matrix}{K [ M ]}_{T} + n + {K [ L ]}_{T} n \pm \\ \sqrt{{( {K [ M ]}_{T} + n + {K [ L ]}_{T} n )}^{2} - 4 {{{K}^{2} [ L ]}_{T} [ M ]}_{T} n}\end{matrix}}{2 Kn} \qquad ( 5 )$$

The sign in front of the square root in eqn. (5) is always required to be negative in order to obtain a physically meaningful answer. If the positive sign is calculated, [SL] is greater than the maximum possible concentration of bound ligand.

The derivative of the physically meaningful [SL] with respect to [L]_{T} is evaluated

$$\frac{d [ SL ]}{d {[ L ]}_{r}} = \frac{1}{2} - \frac{n / K + {[ L ]}_{T} n - {[ M ]}_{T}}{2 \sqrt{{( {[ M ]}_{T} + n / K + {[ L ]}_{T} n )}^{2}} - {{4 [ L ]}_{T} [ M ]}_{T} n} \qquad ( 6 )$$

The heat with respect to total concentration of ligand is expressed as follows:

$$\frac{d Q}{d {[ L ]}_{T}} = - V Δ H \frac{d [ SL ]}{d {[ L ]}_{T}} \qquad ( 7 )$$

where, V is the volume of the reaction cell of the calorimeter and ΔH is the molar enthalpy of binding.

Competitive Binding Site Model

The competitive binding site model can be used to identify the low-affinity binding properties by displacement with moderate-affinity binding system or to identify the high-affinity binding properties by displacement with moderate-affinity binding system because of the limitation associated with accessible value of K when using the incremental titration method.

FIG. 2A demonstrates two different ligands may require a different number of binding subunits in order to bind to a receptor. This suggests that the binding stoichiometry varies depending on the ligands, L₁ and L₂. Therefore, the two distinct ligands equilibrated with binding sites can be expressed as S₁+L₁ [symbol] S₁L₁ and S₂+L₂ [symbol] S₂L₂, where S₁ and S₂ are the binding sites consisting of n- and m-numbers of binding subunits, and [S₁L₁] and [S₂L₂] are the concentration of the bound L and L₂, respectively.

The binding equilibrium constants are expressed as

$${K}_{1} = \frac{[ {S}_{1} {L}_{1} ]}{[ {S}_{1} ] [ {L}_{1} ]} \\ {K}_{2} = \frac{[ {S}_{2} {L}_{2} ]}{[ {S}_{2} ] [ {L}_{2} ]} \qquad ( 8 )$$

The total conservation of receptor and ligands whose binding stoichiometries are n and m, respectively, is shown below.

[M]_{T}=[M]+n[S₁L₁]+m[S₂L₂]

[L₁]_{T}=[L₁]+[S₁L₁]

[L₂]_{T}[L₂L₂]+[S₂L₂] (9)

where [M]_{T}, [L₁]_{T}, and [L₂], are the total concentrations of M, L₁, and L₂, respectively. The amount of heat released with respect to increasing [L₁]_{T} is shown below.

$$\frac{d Q}{d {[ {L}_{1} ]}_{T}} = - V ( Δ {H}_{1} \frac{d [ {S}_{1} {L}_{1} ]}{d {[ {L}_{1} ]}_{T}} + Δ {H}_{2} \frac{d [ {S}_{2} {L}_{2} ]}{d {[ {L}_{1} ]}_{T}} ) \qquad ( 10 )$$

Two different solutions for [S₁L₁] and [S₂L₂] are required to calculate the heat released according to eqn. (10). Rearranging eqns. (8-10) yields two cubic equations which are a function of both [S₁L₁] and [S₂L₂], respectively:

A[S₁L₁]³+B[S₁L₁]²+C[S₁L₁]+D=0 (11)

E[S₂L₂]³+F[S₂L₂]²+G[S₂L₂]+H=0 (12)

where the coefficients of eqns. (11) and (12) are given by eqn. (S14).

Eqns. (11) and (12) have three real roots (α, β, and γ) and only one root yields a physically meaningful answer. For example, the real solution of eqn. (11) is γ for titration with a high-affinity ligand and β for a low-affinity ligand, respectively. In contrast, the solution for eqn. (12) is β for a high-affinity ligand and γ for a low-affinity ligand. The physically meaningful root among the three real roots of eqn. (11) is required to satisfy two constraints simultaneously. The first constraint is the value of the concentration of the bound ligand is positive ([S₁L₁]>0). [S₁L₁] must be less than [L₁]_{T} when [L₁]_{T} is less than [M]_{T}/n because the concentration of bound ligand cannot be excess than the concentration of injected ligand. [S₁L₁] must be less than [M]_{T}/n when [L₁]_{T} is larger than [M]_{T}/n because the concentration of bound ligand cannot be in excess of [S₁]_{T}. Therefore, the second constraint is that [S₁L₁] is less than the maximum feasible value, which is the smallest among the two numbers between [L₁]_{T} and the concentration of fully saturated bound ligand ([M]_{T}/n). In the same manner, the physically meaningful root of eqn. (12) requires that [S₂L₂] is positive and less than both [M]_{T}/m and [L₂]_{T}.

After selecting the root for the cubic equation for the concentration of the bound ligand, the derivative of both [S₁L₁] and [S₂L₂] with respect to L₁ corresponding to the physically meaningful root can be obtained. Note that L₁ is the ligand added into the sample cell in order to develop the following derivations of equation for the binding isotherm and their parameters. The parameters to obtain the differential forms of the bound ligands are given by eqn. (S15).

Two Independent Binding Sites Model

Two different binding sites in which an identical ligand binds with different affinity to the same receptor, M, (See FIG. 1B) may have different binding configurations, meaning that the binding stoichiometry can vary depending on ligands. Therefore, the two different binding reactions can be expressed as S₁+L₁ [symbol] S₁L and S₂+L [symbol] S₂L, where S₁ and S₂ are the binding sites, and [S₁L] and [S₂L] are the concentration of the bound L with S₁ and bound L with S₂, respectively. The binding equilibrium constants are given by

$${K}_{1} = \frac{[ {S}_{1} L ]}{[ {S}_{1} ] [ L ]} \\ {K}_{2} = \frac{[ {S}_{2} L ]}{[ {S}_{2} ] [ L ]} \qquad ( 13 )$$

The concentration of S₁ and S₂ may be different. Thus, the concentration of each binding subunit should be considered separately, i.e. [M₁]_{T} and [M₂]_{T}. The total conservation of receptors with binding stoichiometry, n and in, and a ligand are given by

[M₁]_{T}=[M₁]+n[S₁L]

[M₂]_{T}=[M₂]+m[S₂L]

[L]_{T}=[L]+[S₁L]+[S₂L] (14)

The amount of heat released with respect to increasing [L]_{T} is shown below.

$$\frac{d Q}{d {[ L ]}_{T}} = - V ( Δ {H}_{1} \frac{d [ {S}_{1} L ]}{d {[ L ]}_{T}} + Δ {H}_{2} \frac{d [ {S}_{2} L ]}{d {[ L ]}_{T}} ) \qquad ( 15 )$$

Two different solutions for [S₁L] and [S₂L] are required to calculate the heat released according to eqn. (15). Rearranging eqns. (13) and (14) yields two cubic equations as a function of [S₁L] and [S₂L], respectively:

A[S₁L]³+B[S₁L]²+C[S₁L]+D=0 (16)

E[S₂L]³+F[S₂L]²+G[S₂L]+H=0 (17)

where the coefficients are given by eqn. (S16).

Each cubic equation has three real roots but only one physically meaningful answer, similar to the competitive binding site model. The same criteria applied for determining the physically meaningful root for the competitive binding sites model can be applied to the two independent binding sites model (eqns. (16) and (17)). Therefore, the criteria for selecting a realistic value are [S₁L] is positive and less than either [M]_{T}/n or [L]_{T}, and [S₂L] is positive and less than either [M]_{T}/m or [L]_{T}. After selecting the appropriate root for the solution by using the above criteria, the derivatives of both [S₁L] and [S₂L] with respect to [L]_{T} can be used to calculate the heat released by eqn. (15). The coefficients associated with the cubic equations to obtain the derivative forms of the bound ligands are given by eqn. (S17).

Mathematical modeling for real-time estimation of thermodynamic binding parameters in the continuous injection method

An additional advantage of the continuous injection mode is that the data at the inflection point(s) of the binding isotherm is used to determine K, n, and ΔH from the appropriate model. Instead of fitting the data collected by the incremental injection method with an appropriate binding model, thermodynamic binding parameters are obtained by solving a system of algebraic equations during the continuous titration while the incremental injection method requires completion of the experiment before the data can be analyzed.

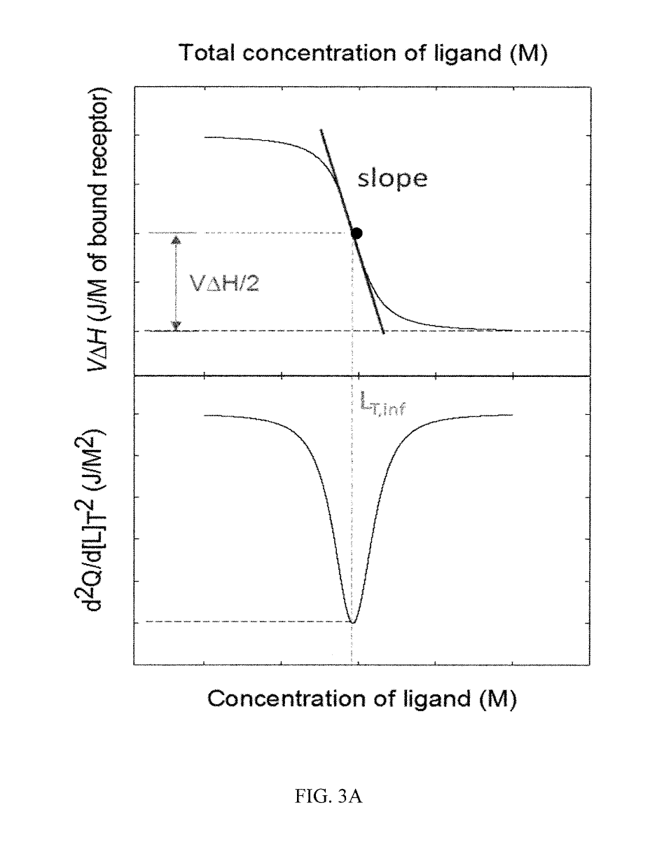

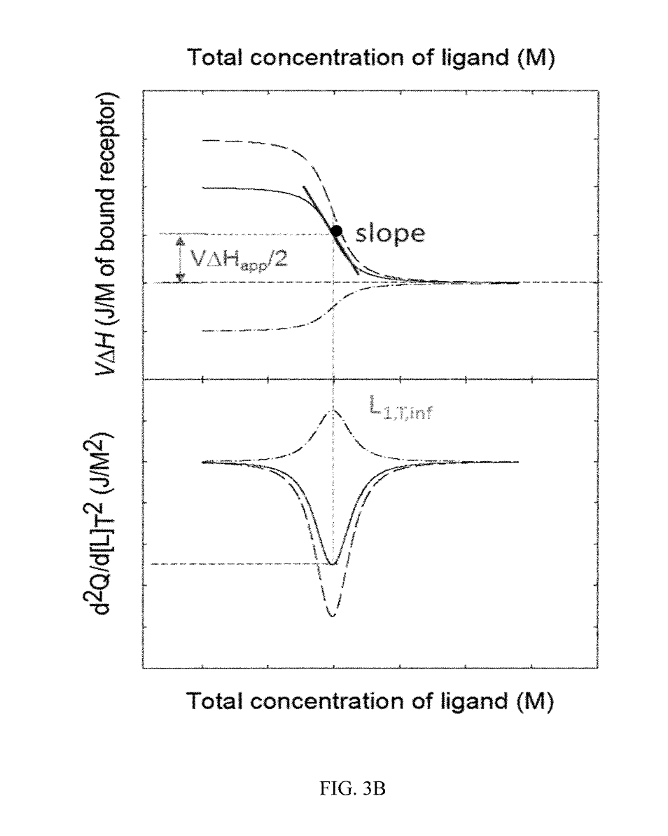

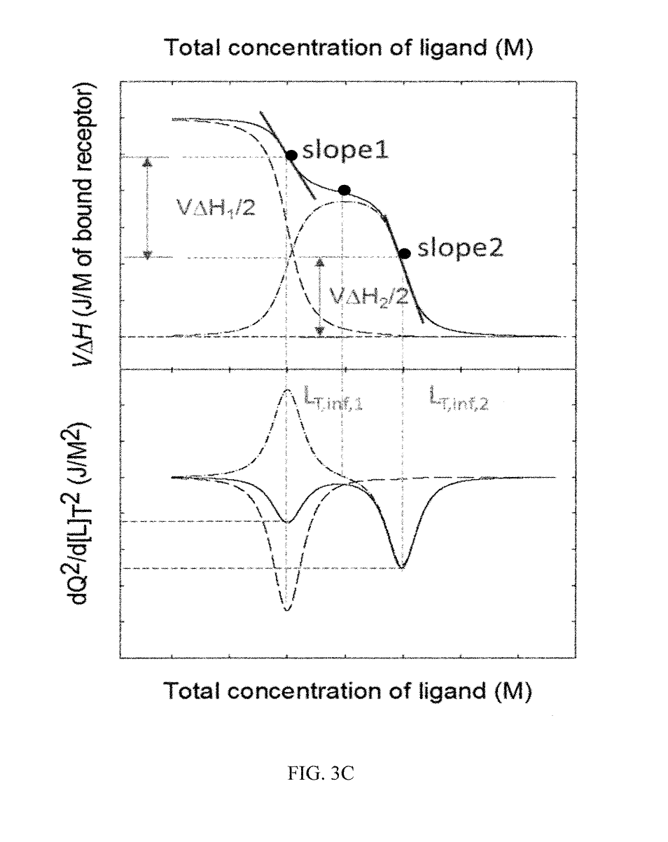

Schematics of real-time evaluation of thermodynamic binding parameters for single independent site binding, competitive binding, and two independent binding sites model are shown in FIGS. 3A, 3B and 3C, respectively. Inflection point(s) on the binding isotherm are marked with a dot.

Single Independent Binding Site Model

FIG. 3A is a schematic of the method used to determine the thermodynamic binding parameters from the binding isotherm with respect to [L]_{T} and its derivative. The binding isotherm for the single independent binding site typically produces a sigmoid. The x-axis value at the inflection point of the binding isotherm for the single independent site binding (eqn. (7)) is obtained from the second derivative of binding isotherm as a function of [L]_{T}, where

$$\frac{{d}^{3} Q}{d {[ L ]}_{T}^{3}} = 0 \qquad ( 18 )$$

Note that the binding isotherm, eqn. (7), is the first derivative of Q with respect to [L]_{T}. The total concentration of ligand at the inflection point, [L]_{T,inf}, becomes

$${[ L ]}_{T , inf} = \frac{{[ M ]}_{T}}{n} - \frac{1}{K} \qquad ( 19 )$$

Substitution of eqn. (19) into eqn. (6), and insert this corresponding result into eqn. (7), the first derivative of Q with respect to [L]_{T} at the inflection point becomes

$$\frac{d Q}{d {[ L ]}_{T}} {\vert}_{{[ L ]}_{T} = {[ L ]}_{T , inf}} = - \frac{V Δ H}{2} \qquad ( 20 )$$

The slope at the inflection point determined from the derivative of eqn. (7) is

$$\frac{{d}^{2} Q}{d {[ L ]}_{T}^{2}} {\vert}_{{[ L ]}_{T} = {[ L ]}_{T , inf}} = - \frac{V Δ H}{4} \sqrt{\frac{Kn}{{[ M ]}_{T}}} \qquad ( 21 )$$

The parameters, K, n, and ΔH, can be obtained by solving the system of algebraic equations, eqns. (19-21). Details of the derivation for eqns. (19-21) are described herein.

Competitive Binding Site Model

FIG. 3B shows the schematic for obtaining the values at the inflection point and deconvolution of the total heat of binding to the individual heat of binding and dissociation due to displacement of the low-affinity ligand. Because one ligand binds as the other ligand dissociates, the apparent binding isotherms are the sum of a binding isotherm of the high-affinity ligand and dissociation of the low-affinity ligand. The binding isotherm for competitive binding depends on the total concentration of the injecting ligand ([L₁]_{T}), and produces a sigmoid similar to single independent binding. The magnitude of the first plateau represents approximately (not exactly because there is a loss of volume by the injection in the constant volume of the calorimeter cell and the injected ligand binds only a part of the receptor depending on the value of K) the sum of the heat of binding of high-affinity binding sites (black dash line) and the heat of dissociation of ligands from low-affinity binding sites (black dash-dot line). The slope at the inflection point between the plateaus represents the relative affinity of the binding site with high-affinity ligand (L₁) to the low-affinity ligand (L₂). For the competitive binding site model, thermodynamic binding parameters for L₂ are obtained independently by using the single independent binding site model. Therefore, K, n, and ΔH for L₁ are the only unknown variables. Analogous to the single independent binding site model, thermodynamic binding parameters for competitive binding sites model are described by the apparent binding constant, Kₐₚₚ, and the apparent heat of binding, ΔHₐₚₚ, yields the system of algebraic equations:

$$\begin{matrix}{[ {L}_{1} ]}_{T , inf} = \frac{{[ M ]}_{T}}{n} - \frac{1}{{K}_{app}} & ( 22 ) \\ \frac{d Q}{d {[ {L}_{1} ]}_{T}} {\vert}_{{[ {L}_{1} ]}_{r} = {[ {L}_{1} ]}_{r , inf}} = - \frac{V Δ {H}_{app}}{2} & ( 23 ) \\ \frac{{d}^{2} Q}{d {[ {L}_{1} ]}_{T}^{2}} {\vert}_{{[ {L}_{1} ]}_{r} = {[ {L}_{1} ]}_{r , inf}} = - \frac{V Δ {H}_{app}}{4} \sqrt{\frac{{K}_{app} n}{{[ M ]}_{T}}} & ( 24 )\end{matrix}$$

where, Kₐₚₚ is defined as

$${K}_{app} = \frac{{K}_{1}}{1 + {K}_{2} [ {L}_{2} ]} \qquad ( 25 )$$

and ΔHₐₚₚ is defined as

$$Δ {H}_{app} = Δ {H}_{1} - \frac{n}{m} Δ {H}_{2} \frac{{K}_{2} [ {L}_{2} ]}{1 + {K}_{2} [ {L}_{2} ]} \qquad ( 26 )$$

Since [L₂] is unmeasurable, [L₂] is approximated as [L₂]T in eqn. (25) and (26). This assumption leads to estimations of K₁ and ΔH₁ by eqns. (22-26) to be larger than the values evaluated by the ITC equation for the competitive binding site model, eqn. (10).

Two Independent Binding Sites Model

FIG. 3C demonstrates how data at inflection points and the individual heats of binding associated with each binding site for a system consisting of two independent binding sites is obtained. The ligand binding is favorable to the high-affinity binding sites rather than the low-affinity binding sites when both binding sites are not bound. The magnitude of the first plateau represents approximately (due to loss of receptor by injection and presence of unbound ligand depending on the value of K) the heat of binding of the high-affinity binding sites of the receptors with ligands. After the high-affinity binding site is almost saturated ([S₁]_{T}≈[L]_{T}), ligands bind with the low-affinity binding site. The magnitude of the second plateau represents approximately the heat of binding of the low-affinity binding sites of the receptors with ligands since binding of low affinity site dominates after the high-affinity binding sites are saturated. The binding isotherm after [S₁]_{T}=[L]_{T} is similar to the binding isotherm of the single independent binding site for the low-affinity binding site. Therefore, the binding isotherm of two independent binding sites model shows a double sigmoid. The slope of the first inflection point (slope 1) between the first two plateaus represents the relative affinity of the high-affinity binding site to the low-affinity binding site with the ligand because ligands begin to bind the low-affinity binding site. The second inflection point located on the second plateau does not contain useful information for the determination of thermodynamic binding parameters. The slope of the third inflection point (slope 2) between the second and third plateaus represents the affinity of the low affinity binding site with the ligand. Since the ligands bind with high- and low-affinity binding site competitively, the values at the first inflection point are apparent values. However, binding at high-affinity binding sites doesn't influence the characteristic binding isotherm of low-affinity binding site because the high-affinity binding site is almost saturated when Kₐₚₚ is larger than 10⁴ M⁻⁴ as shown in FIG. 3C that the overall binding isotherm and the binding isotherm for low-affinity binding sites overlap. The algebraic equations (eqns. (22-25)) of the competitive binding site model are used for the range from the first plateau to the second plateau by shifting of the binding isotherm with the amount of the heat of binding for L₂(ΔH₂) on the y-axis. The same algebraic equation (eqns. (19-21)) of the single independent binding site model are used for the low-affinity binding sites by shifting of the binding isotherm on the x-axis to the point where all of the high-affinity binding sites are saturated. Due to presence of two independent binding sites, six binding parameters are determined from the algebraic equations. The parameters can be solved with eqns. (27-33).

The value of the x-axis at the first inflection point of the binding isotherm, [L]_{T,inf,1}, is identical to eqn. (22).

$${[ L ]}_{T , inf , 1} = \frac{{[ {M}_{1} ]}_{T}}{n} - \frac{1}{{K}_{app}} \qquad ( 27 )$$

The value of the x-axis at the third inflection point of the binding isotherm, [L]_{T,inf,2}, is identical to eqn. (19) with a shift of the amount of [S]_{T}(=[M₁]_{T}/n).

$${[ L ]}_{T , inf , 2} - \frac{{[ {M}_{1} ]}_{T}}{n} = \frac{{[ {M}_{2} ]}_{T}}{m} - \frac{1}{{K}_{2}} \qquad ( 28 )$$

The value of the y-axis at the first inflection point of the binding isotherm is similar to eqn. (24) with binding of ligand to the low-affinity binding site instead of dissociation of the low affinity ligand.

$$\frac{d Q}{d {[ {L}_{1} ]}_{T}} {\vert}_{{[ L ]}_{T} = {[ L ]}_{T , inf , , 1}} = - \frac{V ( Δ {H}_{1} + Δ {H}_{2} )}{2} \qquad ( 29 )$$

The value of the y-axis at the third inflection point of the binding isotherm is identical to the equation for the single independent binding site model, eqn. (20).

$$\frac{d Q}{d {[ {L}_{1} ]}_{T}} {\vert}_{{[ L ]}_{T} = {[ L ]}_{T , inf , , 2}} = - \frac{V Δ {H}_{2}}{2}$$

Analogous to the competitive binding site model, the slope at the first inflection point of the isotherm is similar to eqn. (24) using the difference of the magnitude of heat between the first and second plateau (ΔH₁-ΔH₂) instead of ΔHₐₚₚ (the difference of the magnitude of heat between the first and second plateau in the competitive binding site model)

$$\frac{{d}^{2} Q}{d {[ {L}_{1} ]}_{T}^{2}} {\vert}_{{[ L ]}_{T} = {[ L ]}_{T , inf , 1}} = - \frac{V ( Δ {H}_{1} - Δ {H}_{2} )}{4} \sqrt{\frac{{K}_{app} n}{{[ {M}_{1} ]}_{T}}} \qquad ( 31 )$$

where the apparent binding equilibrium binding constant Kₐₚₚ is

$${K}_{app} = \frac{{K}_{1}}{1 + {K}_{2} [ {M}_{2} ] / m} \qquad ( 32 )$$

The slope at the third inflection point of the isotherm is identical to eqn. (21)

$$\frac{{d}^{2} Q}{d {[ L ]}_{T}^{2}} {\vert}_{{[ L ]}_{T} = {[ L ]}_{T , inf , 2}} = - \frac{V ( Δ {H}_{2} )}{4} \sqrt{\frac{{K}_{2} m}{{[ {M}_{2} ]}_{T}}} \qquad ( 33 )$$

Since the concentration of unbound low-affinity binding site is not measurable, [M₂] is approximated as [M₂]T in eqn. (32). This assumption causes the estimation of K₁ by solving eqns. (31-33) to be larger than K₁ evaluated by ITC equation for two independent binding sites model, eqn. (15).

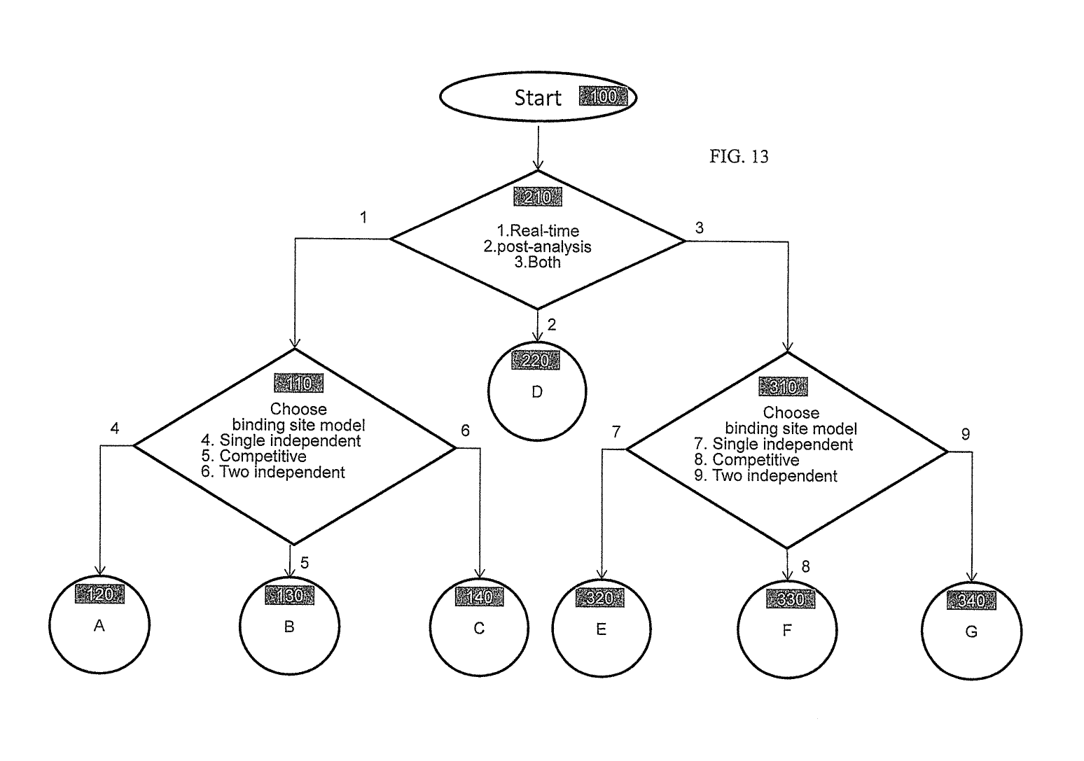

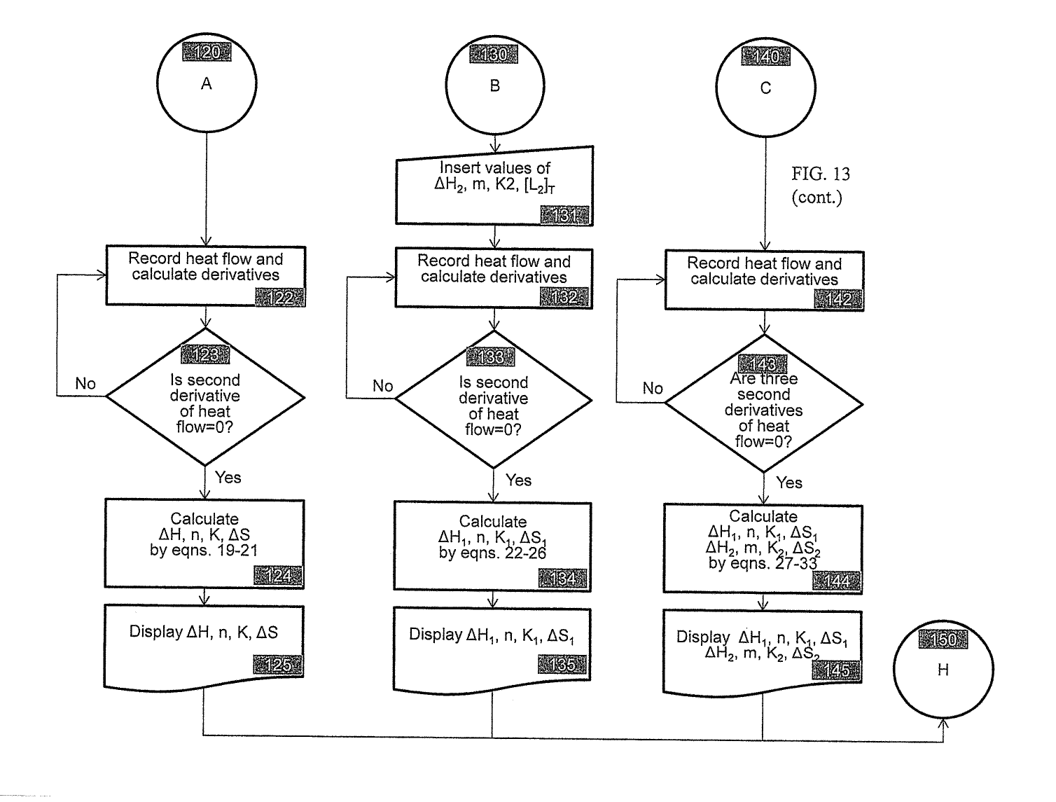

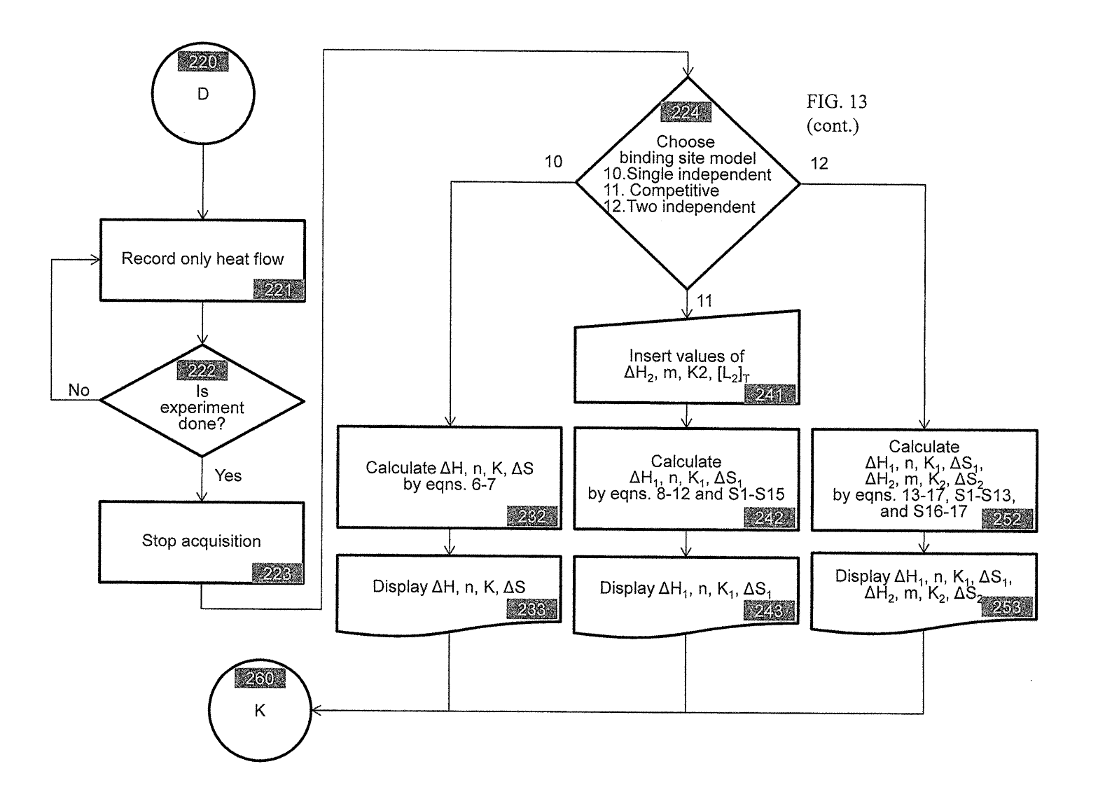

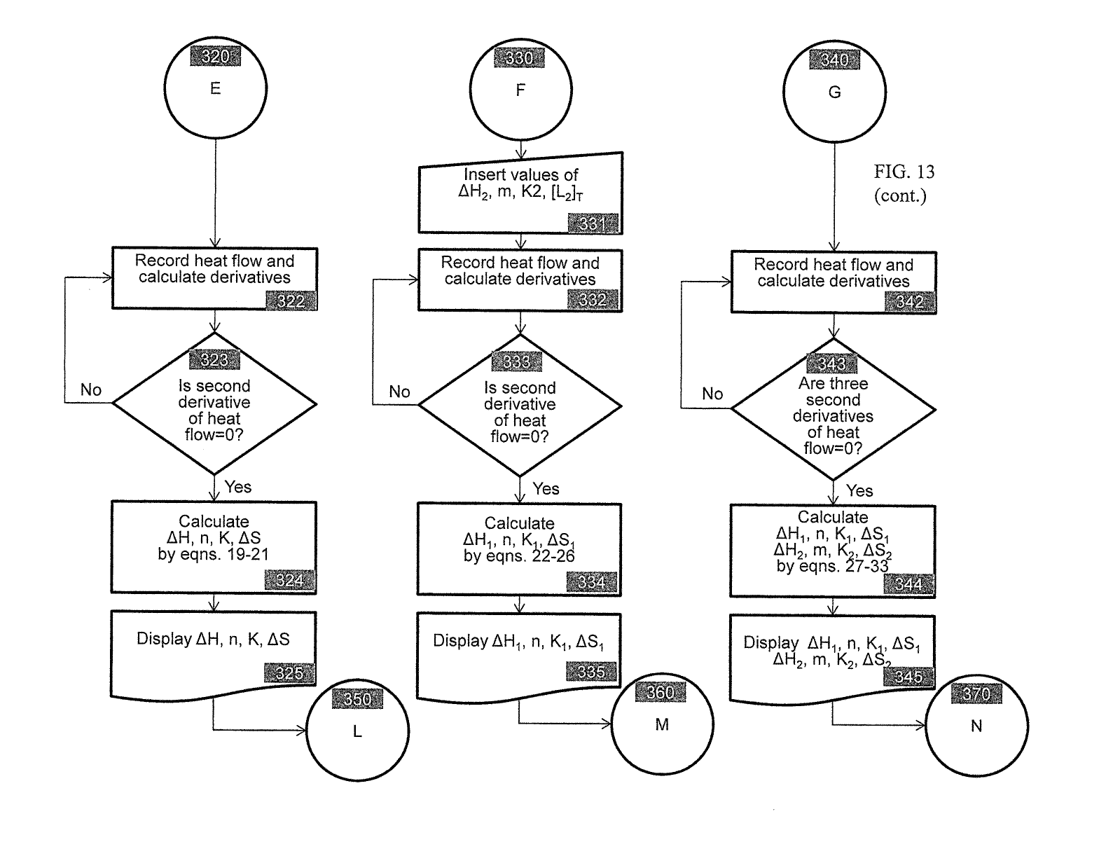

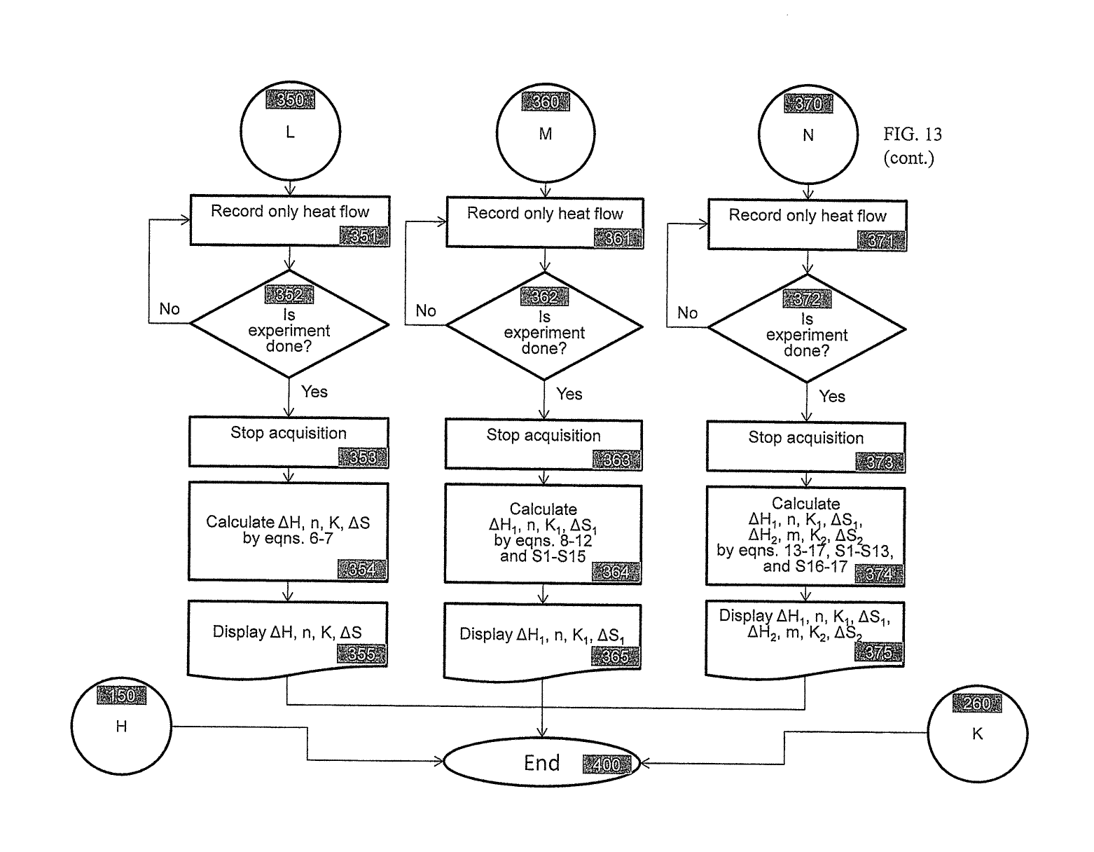

Methods 100, are shown according to aspects of the present invention illustrated in FIG. 13, wherein a user selects the type of analysis to be performed, 210, real-time analysis (1), post-analysis (2) or both (3).

Where the user selects real time analysis (1), a further selection 110 is made between types of binding model to be analyzed, single independent binding (4), competitive binding (5) or two independent binding sites (6).

For real time analysis of single independent binding (4), method 120 (A) is followed, including recording heat flow values and calculation of derivatives 122, and determining 123 if the second derivative of heat flow is zero, if not zero, repeat 122 and 123. When the second derivative of heat flow is determined 123 to be zero, calculation 124 of one or more of ΔH, n, K, ΔS is performed using equations 19-21. Calculated values for one or more of ΔH, n, K, ΔS is then displayed 125 to the user. 150 (H) is a page connector illustrating the end 400 the process.

For real time analysis of competitive binding (5), method 130 (B) is followed, including input 131 of ΔH₂, m, K₂ and [L₂]T, recording heat flow values and calculation of derivatives, 132, and determining 133 if the second derivative of heat flow is zero, if not zero, repeat 132 and 133. When the second derivative of heat flow is determined 133 to be zero, calculation 134 of one or more of ΔH₁, n, K₁ and ΔS₁ is performed using equations 22-26. Calculated values for one or more of ΔH₁, n, K₁ and ΔS₁ is then displayed 135 to the user. 150 (H) is a page connector illustrating the end 400 the process.

For real time analysis of two independent binding sites (6), method 140 (C) is followed, recording heat flow values and calculation of derivatives, 142, and determining 143 if the second derivatives of heat flow are zero, if not zero, repeat 142 and 143. When the second derivative of heat flow is determined 143 to be zero, calculation 144 of one or more of ΔH₁, n, K₁ and ΔS₁ and one or more of ΔH₂, m, K₂ and ΔS₂ is performed using equations 27-33. Calculated values for one or more of ΔH₁, n, K₁ and ΔS₁ and one or more of ΔH₂, m, K₂ and ΔS₂ is then displayed 145 to the user. 150 (H) is a page connector illustrating the end 400 the process.

Where the user selects post-analysis (2), process 220 (D) is followed including 221 recording heat flow values and not calculating derivatives. When it is determined 222 that the experiment is finished, acquisition of heat flow values is stopped 223 and the user makes a selection 224 between types of binding model to be analyzed, single independent binding (10), competitive binding (11) or two independent binding sites (12).

In a post-analysis process in which single independent binding (10) is selected, calculation 232 of one or more of ΔH, n, K and ΔS is performed using equations 6-7 and the resulting calculated ΔH, n, K and ΔS is displayed 233. 260 (K) is a page connector illustrating the end 400 the process.

In a post-analysis process in which competitive binding (11) is selected, values for one or more of ΔH₂, m, K₂ and [L₂]_{T} is input 241, calculation 242 of one or more of ΔH₁, n, K₁ and ΔS₁ is performed using equations 8-12 and S1-S15 and the resulting calculated one or more of ΔH₁, n, K₁ and ΔS₁ is displayed 243. 260 (K) is a page connector illustrating the end 400 the process.

In a post-analysis process in which two independent binding sites (12) is selected, calculation 252 of one or more of ΔH₁, n, K₁ and ΔS₁ and one or more of ΔH₂, m, K₂ and ΔS₂ is performed using equations 13-17, S1-S13 and S16-S17 and the resulting calculated one or more of ΔH₁, n, K₁ and ΔS₁ and one or more of ΔH₂, m, K₂ and ΔS₂ is displayed 253. 260 (K) is a page connector illustrating the end 400 the process.

Where the user selects both real time analysis and post-analysis (3), a further selection 310 is made between types of binding model to be analyzed, single independent binding (7), competitive binding (8) or two independent binding sites (9).

For real time analysis and post-analysis of single independent binding, method 320 (E) is followed, including recording heat flow values and calculation of derivatives 322, and determining 323 if the second derivative of heat flow is zero, if not zero, repeat 322 and 323. When the second derivative of heat flow is determined 323 to be zero, calculation 324 of one or more of ΔH, n, K, ΔS is performed using equations 19-21. Calculated values for one or more of ΔH, n, K, ΔS is then displayed 325 to the user. Page connector 350 (L) shows continuation of the process including recording 351 heat flow values and not calculating derivatives, determination 352 whether the experiment is finished and if so, acquisition of heat flow values is stopped 353 and calculation 354 of one or more of ΔH, n, K and ΔS is performed using equations 6-7. The resulting calculated ΔH, n, K and ΔS is displayed 355 and the process is ended 400.

For real time analysis and post-analysis of competitive binding, method 330 (F) is followed, including input of 331 of one or more of ΔH₂, m, K₂ and [L₂]_{T}, recording heat flow values and calculation of derivatives 332, and determining 333 if the second derivative of heat flow is zero, if not zero, repeat 332 and 333. When the second derivative of heat flow is determined 333 to be zero, calculation 334 of one or more of ΔH₁, n, K₁ and ΔS₁ is performed using equations 22-26. Calculated values for one or more of ΔH₁, n, K₁ and ΔS₁ is then displayed 335 to the user. Page connector 360 (M) shows continuation of the process including 361 recording heat flow values and not calculating derivatives, determination 362 whether the experiment is finished and if so, acquisition of heat flow values is stopped 363 and calculation 364 of one or more of ΔH₁, n, K₁ and ΔS₁ is performed using equations 8-12 and S1-S15. The resulting calculated ΔH₁, n, K₁ and ΔS₁ is displayed 365 and the process is ended 400.

For real time analysis and post-analysis of single independent binding, method 340 (G) is followed, including recording heat flow values and calculation of derivatives 342, and determining 343 if the second derivative of heat flow is zero, if not zero, repeat 342 and 343. When the second derivative of heat flow is determined 343 to be zero, calculation 344 of one or more of ΔH₁, n, K₁ and ΔS₁ and one or more of ΔH₂, m, K₂ and ΔS₂ is performed using equations 27-33. Calculated values for one or more of ΔH₁, n, K₁ and ΔS₁ and one or more of ΔH₂, m, K₂ and ΔS₂ is then displayed 345 to the user. Page connector 370 (N) shows continuation of the process including recording 371 heat flow values and not calculating derivatives, determination 372 whether the experiment is finished and if so, acquisition of heat flow values is stopped 373 and calculation 374 of one or more of ΔH₁, n, K₁ and ΔS₁ and one or more of ΔH₂, m, K₂ and ΔS₂ is performed using equations 13-17, S1-S13 and S16-S17. The resulting calculated one or more of ΔH₁, n, K₁ and ΔS₁ and one or more of ΔH₂, m, K₂ and ΔS₂ is displayed 355 and the process is ended 400.

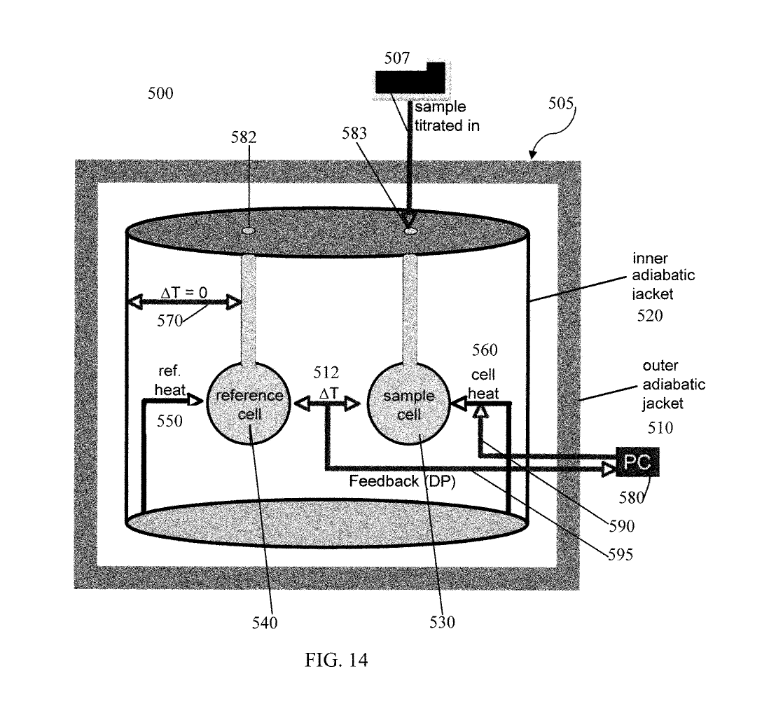

FIG. 14 is a schematic diagram of a system 500 according to aspects of the present invention including an isothermal titration calorimeter in signal communication with a computer 580, wherein the computer runs a computer program for determining one or more binding characteristics of a ligand and a receptor using heat flow values obtained by an isothermal titration calorimetry method, the computer program operative to calculate the one or more binding characteristics in real-time, producing one or more determined binding characteristics displayed to a user. The isothermal titration calorimeter includes a sample cell 530 and a reference cell 540 wherein a difference in temperature 512 between the sample cell heat 560 and reference cell heat 550 is detected by thermopiles and communicated 595 to the computer in order to maintain ΔT=0 by controlling cell heat 560 with electric signal 580. Sample cell heat 560 and reference cell heat 550 are monitored by PC 580 and the difference heat between sample cell heat and reference cell heat is heat flow. The computer is operably connected to a heat input apparatus and directs the input of heat 590 to the sample cell as needed. Portals 583 and 582 are present for introduction and removal of materials into and out of the sample cell 530 and reference cell 540. An optional syringe pump for continuous injection of a ligand into the sample cell of the isothermal titration calorimeter is shown schematically at 507.

Embodiments of inventive compositions and methods are illustrated in the following examples. These examples are provided for illustrative purposes and are not considered limitations on the scope of inventive compositions and methods.

### EXAMPLES

### Example 1

Experimental validation of methods of determining one or more binding characteristics using ITC, where [L]_{T} is known, for a single competitive binding site or two independent binding sites model are provided herein. For competitive binding site model, the titration of a mixture of Ba²⁺ and ethylenediaminetetraacetic acid (EDTA; receptor) in the calorimeter cell with Ca²⁺ from the syringe (ligand) is used. A receptor with two different binding sites can be experimentally simulated by mixing two different molecules which have different thermodynamic binding properties to the ligand and therefore demonstration of methods according to aspects of the invention as applied to the two independent binding site model was conducted by titrating ethylenediaminetetraacetic acid (EDTA) and 1,3-diaminopropane-N,N,N′,N′-tetraacetic acid (DPTA) mixture with Ca²⁺. Values at the inflection point(s) were used to evaluate the thermodynamic binding parameters for each binding model during experimental titrations. Computational simulations were used to estimate the accuracy of the real-time evaluation of thermodynamic binding parameters at various K, n, and ΔH.

Materials

All materials were used without any further purification. Ethylenediaminetetraacetic acid (EDTA), 1,3-diaminopropane-N,N,N′,N′-tetraacetic acid (DPTA), barium chloride, and N-(2-Hydroxy-1,1-bis(hydroxymethyl)ethyl)glycine (Tricine) were obtained from Sigma-Aldrich. Calcium nitrate tetrahydrate, and sodium hydroxide were obtained from Alfa Aesar. 4-(2-hydroxyethyl)-1-piperazineethanesulfonic acid (HEPES) was obtained from Research Organics, Inc.

Sample Preparation

Buffer solutions, EDTA, EGTA, Ca(NO₃)₂, and BaCl₂ were prepared in Tricine or HEPES. The pH of these solutions was adjusted to 8.5 with 1 M NaOH solution. For the single independent binding site and two independent binding sites experiments, Tricine (20 mM) was used as a buffer solution. For the competitive binding site experiment, HEPES (20 mM) was used as a buffer solution.

Isothermal Titration Calorimetry

The titrations were performed on a NanoITC (TA Instruments, New Castle, Del.) with gold cells with a cell volume of 1 mL. ITC experiments were carried out at 25° C. and the stirring speed was 250 rpm. For the single independent binding experiment, ligand of 20 mM in 100 μL syringe and for the competitive binding site and two independent binding sites model, ligand of 10 mM in 250 μL syringe were used. The injection rate was varied 1.7-10 μL/min for the continuous injection mode under setting tab in ITCRun software. During the equilibration, mass transfer of ligand at the tip of the injection syringe reduces the integrated heat of the first injection. The first data point for the incremental injection mode and data for the initial 10 μL of injection for the continuous injection mode is excluded for data fitting with binding models due to reduced amount of the heat flow.

Accuracy and Experiment Time Comparison

Binding of Ba²⁺ and EDTA were performed and analyzed with the single independent binding site model in order to obtain the confidence intervals depending on the number of injections.

BaCl₂ (20 mM) located in 100 μL syringe was injected into an EDTA (1 mM)/Tricine buffer solution. The number of injections for the incremental injection experiment was 36 with an injection volume of 2.5 μL. An injection rate of 1.7 μL/min was utilized in the continuous injection experiment. The fit for the continuous injection method was done after 0.1 mM of ligand had been injected.

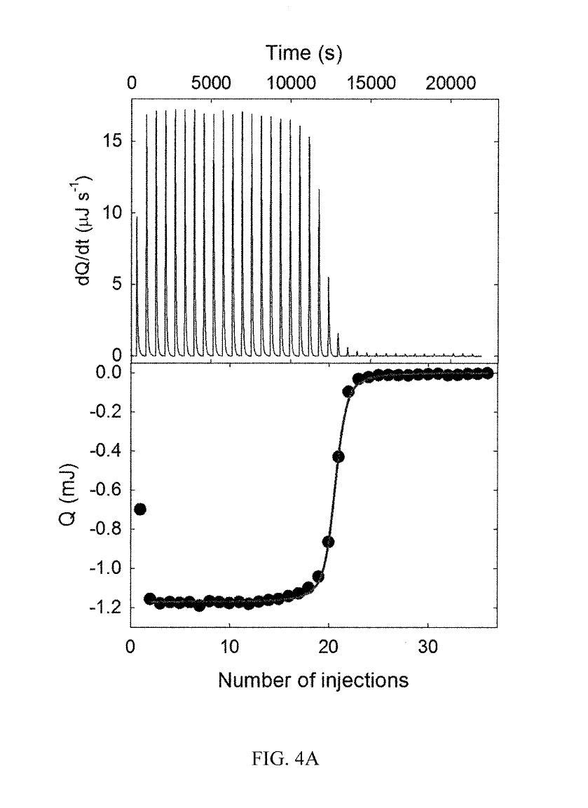

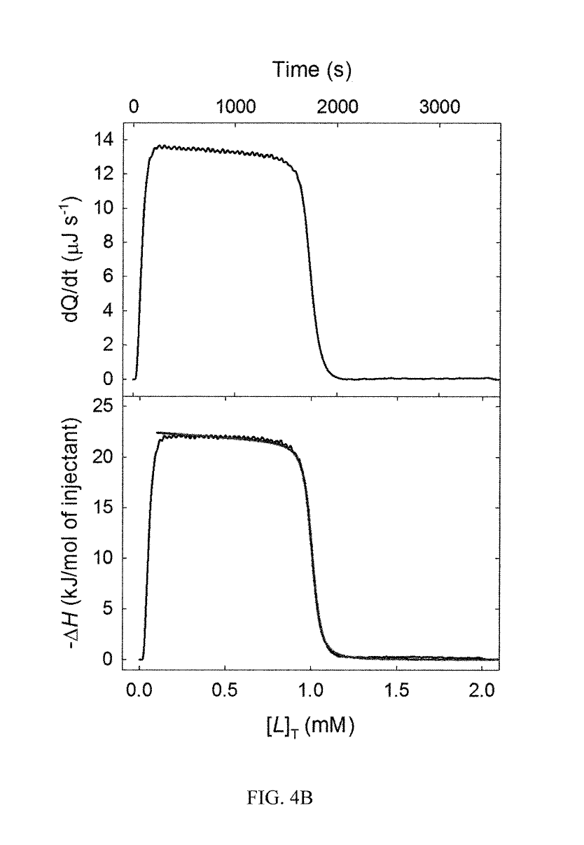

The experimental time for incremental injection mode experiment was ˜6.5 h (36 injections), while the continuous injection experiment completed within ˜1 h. FIG. 4A represents the binding isotherms for binding of Ba²⁺ to EDTA at 25° C. by incremental titration, along with the integrated heats and the corresponding best fit utilizing the single independent binding site model. FIG. 4B represents the raw data for the binding of Ba²⁺ to EDTA at 25° C. using the continuous injection method and the corresponding best fit to the binding isotherm with respect to [L]_{T}. The thermodynamic data are summarized in Table 1 along with the corresponding 99% confidence level range in parentheses.

TABLE 1
Thermodynamic parameters derived from an incremental injection experiments are
dependent on the number of injections (99% confidence level of each thermodynamic properties
are calculated and the ranges of their confidence interval are shown in parentheses)

| number | | | | |
|---|---|---|---|---|
| of | | | | ΔH |
| injections | Experiment | n | ln K | (kJ mol⁻¹) |
| | 1 | 1.001 (0.987, 1.013) | 13.99 (13.4, 14.6) | −23.84 (−23.3, −24.3) |
| 9 | 2 | 0.970 (0.957, 0.985) | 14.26 (13.5, 16.7) | −23.84 (−23.4, −23.2) |
| | 3 | 0.965 (0.945, 0.988) | 14.14 (13.0, 16.5) | −23.28 (−22.3, −24.3) |
| | 1 | 0.985 (0.977, 0.993) | 14.07 (13.6, 14.6) | −23.84 (−23.4, −24.2) |
| 18 | 2 | 1.018 (1.012, 1.024) | 14.05 (13.8, 14.5) | −23.34 (−23.1, −23.7) |
| | 3 | 0.950 (0.940, 0.958) | 13.88 (13.3, 14.8) | −22.80 (−22.3, −23.3) |
| | 1 | 1.079 (1.074, 1.084) | 14.07 (13.8, 14.4) | −23.47 (−23.2, −23.7) |
| 36 | 2 | 1.004 (1.000, 1.008) | 14.13 (13.9, 14.4) | −23.33 (−23.1, −23.6) |
| | 3 | 1.005 (1.001, 1.009) | 14.02 (13.8, 14.3) | −22.8 (−22.6, −23.0) |
| | 1 | 0.9981 (0.9979, 0.9983) | 14.15 (14.13, 14.16) | −21.565 (−21.56, −21.57) |
| Continuous^{b} | 2 | 0.9786 (0.9784, 0.9788) | 14.11 (14.09, 14.03) | −21.792 (−21.79, −21.80) |
| | 3 | 0.9960 (0.9958, 0.9962) | 13.73 (13.71, 13.75) | −22.042 (−22.04, −22.05) |

^{a}BaCl₂ (20 mM) in the 100 μL syringe was injected into 1 mM EDTA in a Tricine (20 mM) buffer solution at pH 8.5 at 25° C.
^{b}Injection rate for the continuous injection mode is 1.7 μL/min.

Increase in the number of injections demonstrates the confidence interval decreases with a fixed confidence level. Therefore, a trade-off between the magnitude of confidence interval and the number of injections (i.e. number of data points) exists; longer experiment times due to large number of injection results in a smaller confidence interval for the incremental injection mode. The continuous injection method accomplishes reduced confidence intervals along with reduced overall experiment time, when compared to the incremental injection method.

Competitive Binding Site Model

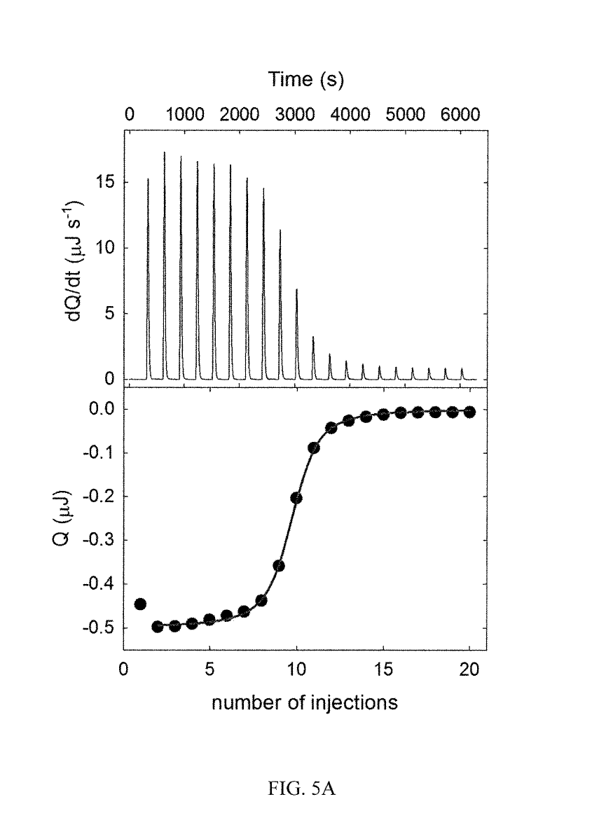

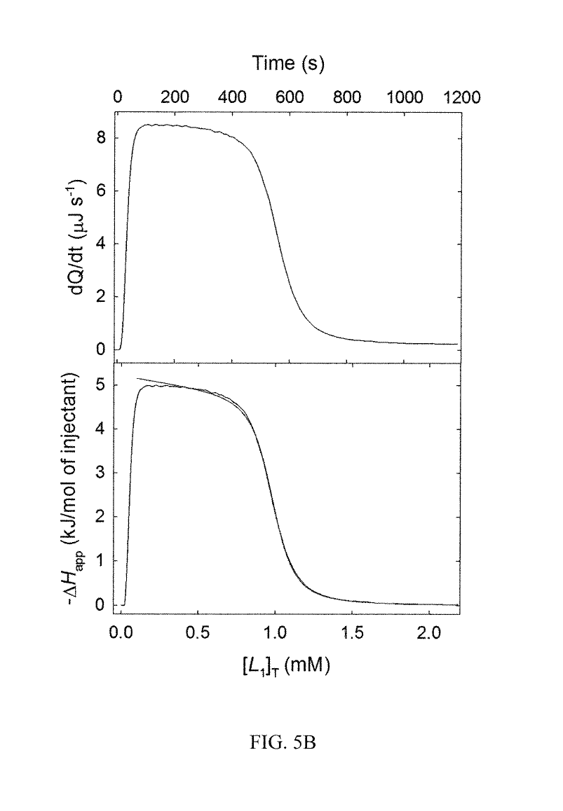

For replicating the competitive binding sites model, Ca²⁺ was titrated into a mixture of Ba²⁺ and EDTA. This experimental set-up allows study the binding of two competing ligands, Ca²⁺ and Ba²⁺, with the receptor, EDTA. Ca(NO₃)₂ (10 mM) in the syringe was injected into the mixture of 1 mM EDTA and 4 mM BaCl₂ in HEPES. The number of injections for the incremental injection mode experiment is 20 with an injection volume of 10 μL. Injection rate for the continuous injection mode is 10 μL/min. The experimental data and fit results obtained from this competitive binding by continuous titration are summarized in FIG. 5 and Table 2, respectively.

The fit for the continuous injection method was done after 0.1 mM of ligand was injected. Comparison of the competitive binding site model using incremental injection or continuous injection mode is shown in FIGS. 5A and 5B, respectively. The positive peaks of the binding isotherms in the raw data (top) represent the exothermic heat of binding.

TABLE 2
Comparison of thermodynamic parameters for competitive binding determined
by incremental and continuous injection method (99% confidence level of
each thermodynamic properties are calculated and the ranges of their confidence
interval are in the parentheses)^{a}

| | | | ΔH |
|---|---|---|---|
| Injection mode | n | ln K | (kJ mol⁻¹) |
| Incremental^{b} | 0.956 (0.949, 0.963) | 20.47 (20.3, 20.6) | −19.66 (−19.4, −19.9) |
| Continuous^{c} | 1.0105 (1.010, 1.011) | 20.535 (20.52, 20.55) | −20.966 (−20.94, −20.99) |

^{a}Ca(NO₃)₂, 10 mM, in the 250 μL syringe was injected into the mixture of 1 mM EDTA and 4 mM BaCl₂ in a HEPES (20 mM) buffer solution at pH 8.5 at 25° C. The thermodynamic binding parameters for Ba²⁺ and EDTA were obtained independently by an incremental injection method and are 1.36 × 10⁶M⁻¹, 1.00, and −14.57 kJ mol⁻¹ for K₂, m, and ΔH₂, respectively.
^{b}Number of injections is 20 with an injection volume of 10 μL.
^{c}Injection rate for the continuous injection mode is 10 μL/min

Due to unnecessary equilibration time between injections, experiment for the continuous injection method is performed in less time. In addition to shortened experimental time, the increased number of data points from 20 (incremental injection method) to ˜1200 (continuous injection method) results in a reduction of the confidence interval by an order of magnitude for all thermodynamic binding parameters between Ca²⁺ and EDTA. Note that the thermodynamic binding parameters for Ba²⁺ and EDTA were obtained independently by incremental injection and are 1.36×10⁶ M⁻¹, 1.00, and −14.57 kJ mol⁻¹ for K₂, mn, and ΔH₂, respectively.

Two Independent Binding Sites Model

By titrating a mixture of EDTA and DPTA with Ca²⁺, a receptor that consists of two different binding characteristics was probed with a cation. By changing the concentration of EDTA and DPTA in the mixture, the various binding stoichiometry of the receptor, n and m, were simulated. The ligand, Ca(NO₃)₂ (5 mM) in the syringe was injected into the mixture of 0.45 mM EDTA and 0.45 mM DPTA in Tricine. The number of injections for the incremental injection mode experiment is 40 with an injection volume of 6 μL. Injection rate for the continuous injection mode is 7.2 μL/min.

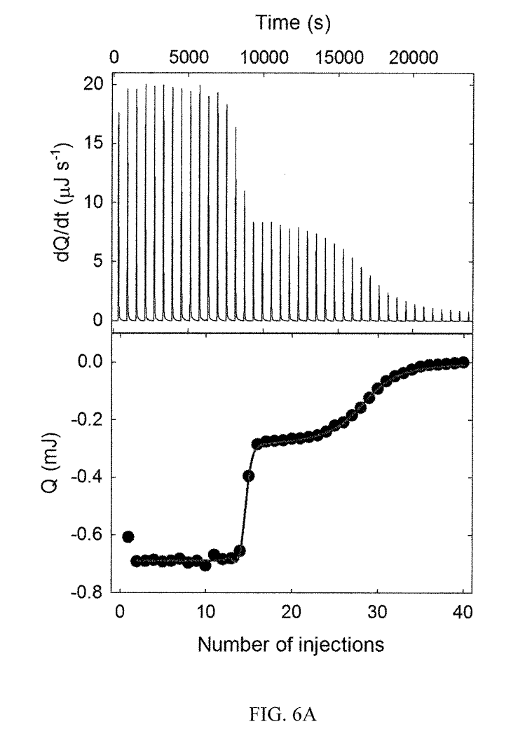

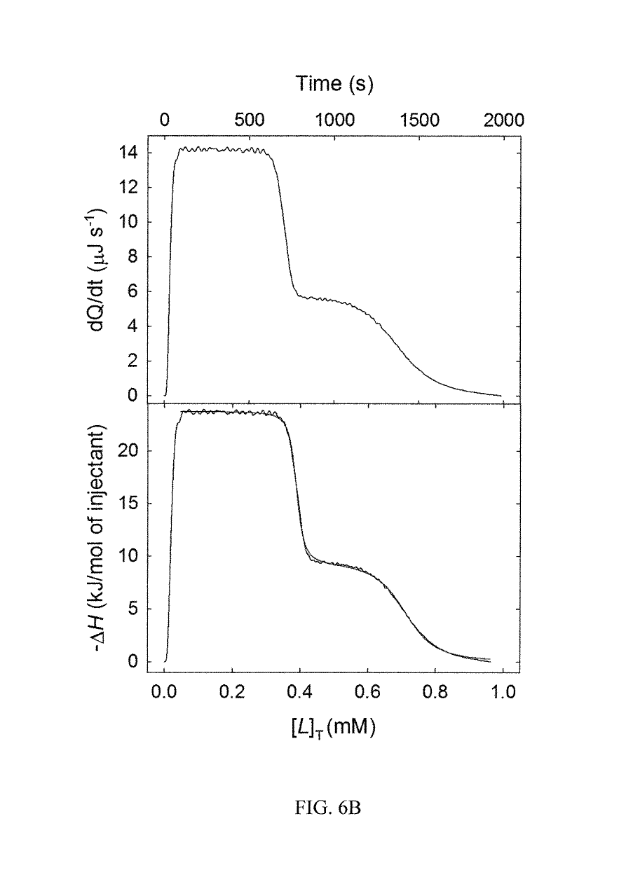

These experiments utilized a receptor (Ca²⁺) which bound with the ligands (EDTA and DPTA) with a stoichiometry (n and in) of unity. The experimental data and fit results obtained from two independent binding sites by continuous titration are summarized in FIG. 6 and Table 3, respectively.

The fit for the continuous injection method was done after 0.05 mM of ligand was injected. Comparison of the two independent binding sites model using incremental injection or continuous injection mode is shown in FIGS. 6A and 6B, respectively. The positive peaks of the binding isotherms in the raw data (top) represent the exothermic heat of binding.

TABLE 3
Comparison of thermodynamic parameters for two independent
binding site model determined by incremental and continuous
injection methods (99% confidence level of each thermodynamic
properties are calculated and the ranges of their confidence
interval are in the parentheses)^{a}

| Incremental^{b} | Injection mode | Continuous^{c} |
|---|---|---|
| 0.968 (0.960, 0.974) | n | 1.022 (1.022, 1.023) |
| 19.50 (18.98, 20.33) | ln K₁ | 18.11 (18.07, 18.15) |
| −23.01 (−22.82, −23.21) | ΔH₁ (kJ mol⁻¹) | −23.81 (−23.78, −23.83) |
| 0.938 (0.913, 0.963) | m | 0.958 (0.956, 0.960) |
| 11.42 (11.14, 11.69) | ln K₂ | 11.27 (11.25, 11.30) |
| −9.31 (−9.01, −9.40) | ΔH₂ (kJ mol⁻¹) | −9.58 (−9.54, −9.62) |

^{a}Ca(NO₃)₂, 5 mM, in the 250 μL syringe was injected into the mixture of 0.45 mM EDTA and 0.45 mM DPTA in a Tricine (20 mM) buffer solution in pH 8.5 at 25° C.
^{b}Number of injections is 40 and each injection volume is 6 μL.
^{c}Injection rate for the continuous injection mode is 7.2 μL/min.

The experimental time was ˜12 times shorter and an order of magnitude reduction was obtained in the confidence intervals for all thermodynamic binding parameters with 99% confidence level by increasing the data point from 40 (incremental injection method) to ˜2000 (continuous injection method).

Computational Simulation for Real-Time Estimation of Thermodynamic Binding Parameters

The cumulative heat, Q for the single independent binding site model can be expressed as

Q=VΔH[SL] (34)

For the regression analysis present in the commercial software used to fit the experimental heats, and the corresponding calculated individual heats associated with the i^{th} injection, ΔQᵢ, a finite difference approximation is used in the commercial software.

ΔQᵢ=Qᵢ−Qᵢ₋₁ (35)

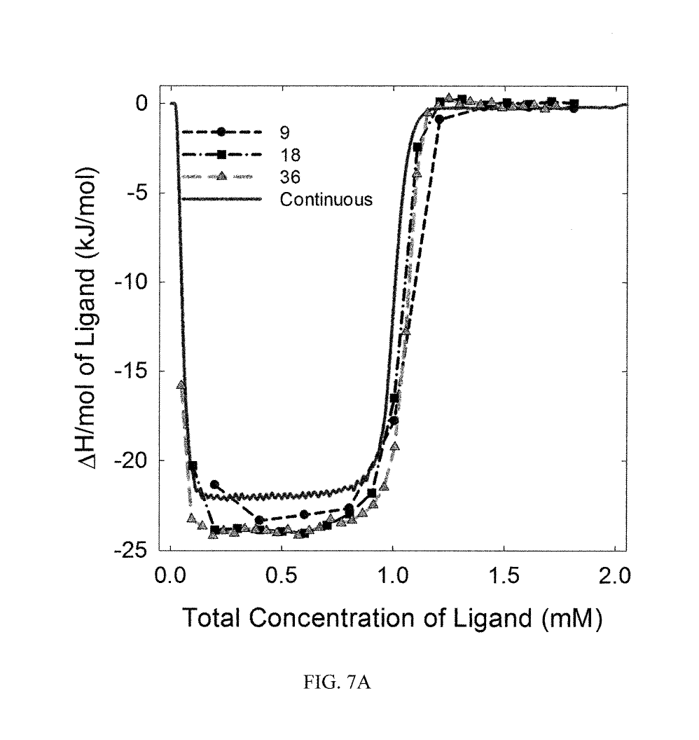

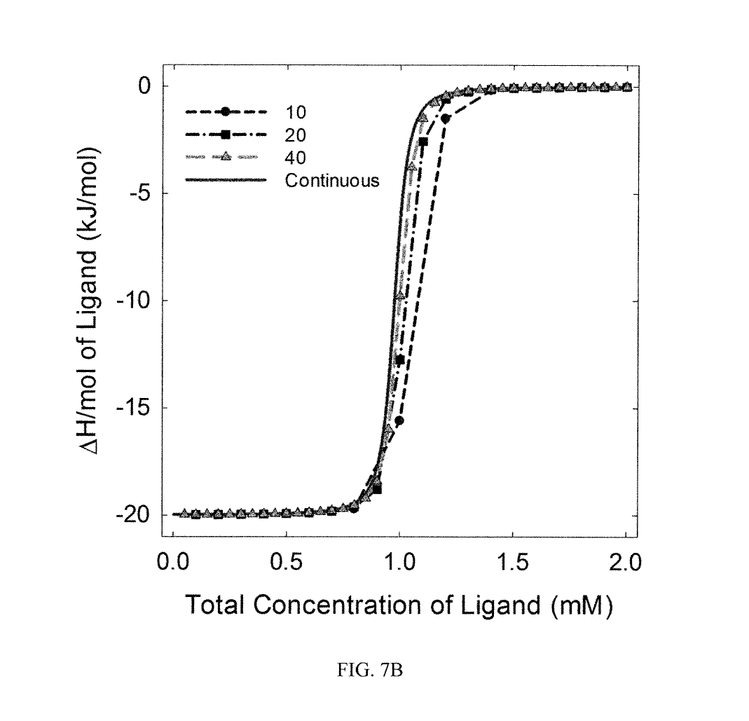

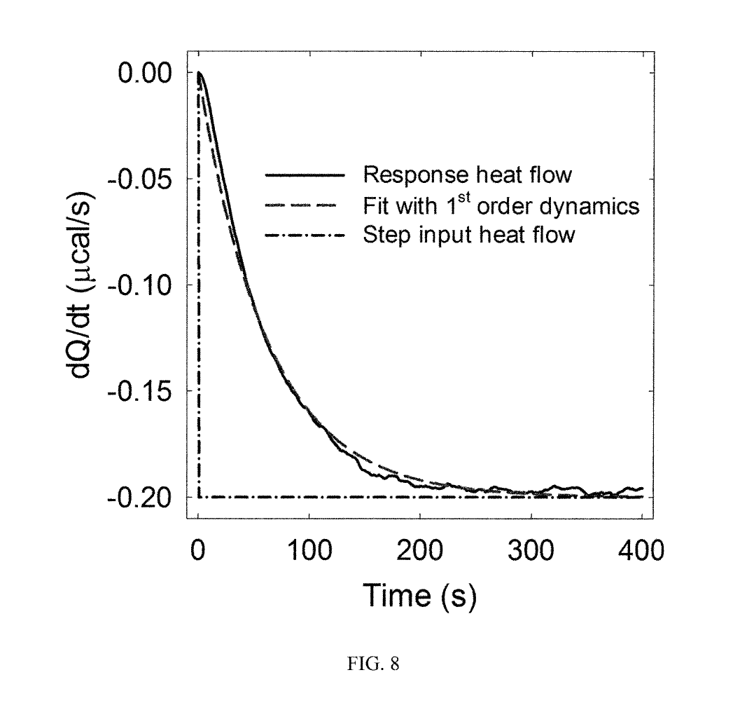

Due to the deviation of differential and finite difference approximation, fitting the curves using eqn. (35) demonstrate shifts in the inflection point and results in different slopes at the inflection point depending on the number of injections. Experimental and simulation results using the differential form and difference of cumulative heat by using a finite difference are shown in FIG. 7.

Comparison of single independent binding model using a differential form of heat and a cumulative heat for independent binding site with various numbers of injections where FIG. 7A shows experiment and FIG. 7B shows simulation (the number in the legend is the number of injections) with 1 mL of total reaction cell, 1 mM of receptor, 20 mM of ligand, K=1×10⁶, n=1, and ΔH=−20 kJ/mol.

The parameters for the simulation are [M]_{T}=1 mM, [L]T=20 mM, K=1×10⁶ M⁻¹, n=1, and ΔH=−20 kJ/mol, and V=1 mL. As the number of injection increases for the incremental injection mode, the inflection point shifts to left and the slope at the inflection point increases. As seen in FIGS. 7A and 7B, the binding isotherm by incremental injection with the higher number of injections approaches the isotherm obtained by the continuous injection method. Both the inflection point of the binding isotherm and the slope at the inflection point have physically meaningful values as shown in eqns. (19-21). Once the inflection point appears in the binding isotherm, the thermodynamic binding parameters ΔH, K, and n can be evaluated prior to completion of the experiment, thereby enabling real-time evaluation of thermodynamic binding parameters.

Thermodynamic binding parameters for simulated single independent binding isotherms (FIG. 3A), values at the inflection point, and calculated thermodynamic binding parameters by solving the algebraic equations for the data at the inflection point are summarized in Table 4.

TABLE 4
Evaluation of thermodynamic parameters at inflection point
for independent binding site model. Injection rate is 2.78 μL/s
and data is acquired every second.

| | | | | | | calculated | | |
|---|---|---|---|---|---|---|---|---|
| model parameters | | | data at inflection point | | | thermodynamic parameters | | |
| | | ΔH | L_{inf} | VΔH/2 | slope | | | ΔH |
| log K | n | (kJ mol⁻¹) | (mM) | (J M⁻¹) | (kJ M⁻²) | log K | n | (kJ mol⁻¹) |
| 5 | 1 | −20 | 0.990 | 10.00 | −50.00 | 5.000 | 1.000 | −20.00 |
| 6 | 1 | −20 | 0.999 | 10.02 | −158.11 | 5.998 | 1.000 | −20.04 |
| 7 | 1 | −20 | 0.999 | 10.20 | −500.00 | 6.983 | 1.000 | −20.40 |
| 8 | 1 | −20 | 1.000 | 10.70 | −1581.10 | 7.941 | 1.000 | −21.40 |
| 6 | ⅓ | −20 | 2.999 | 10.00 | −91.29 | 6.000 | 0.333 | −20.00 |
| 6 | 0.5 | −20 | 1.999 | 10.00 | −111.80 | 6.000 | 0.500 | −20.00 |
| 6 | 2 | −20 | 0.499 | 10.02 | −223.60 | 5.998 | 2.000 | −20.04 |
| 6 | 3 | −20 | 0.332 | 10.04 | −273.86 | 5.996 | 3.001 | −20.08 |
| 6 | 1 | −10 | 0.999 | 5.01 | −79.06 | 5.999 | 1.000 | −10.02 |
| 6 | 1 | −30 | 0.999 | 15.03 | −237.17 | 5.999 | 1.000 | −30.05 |
| 6 | 1 | −40 | 0.999 | 20.03 | −316.23 | 5.999 | 1.000 | −40.06 |

The values from solving eqns. (19-21) gives less than 1% difference for log K and n, and maximum 7% difference for ΔH compared to the simulated values.

For competitive binding site model (FIG. 3B), the binding parameters for a ligand are known from an individual single independent binding site experiment. It is assumed that the thermodynamic properties of binding for the low affinity ligand are known in order to obtain the thermodynamic properties for binding of the high affinity ligand. The values at inflection point for a competitive binding isotherm depend on not only the high affinity binding ligand but also the low affinity binding ligand. In order to reduce the number of simulations, the thermodynamic binding properties of a low affinity ligand were fixed to ΔH₂=−10 kJ/mol and K₂=×10⁴ M⁻¹. The thermodynamic binding parameters for simulation, data at inflection point of the simulated binding isotherm, and the calculated thermodynamic binding parameters by solving the algebraic equations for competitive binding site model are summarized in Table 5.

TABLE 5
Evaluation of thermodynamic parameters at inflection point
for competitive binding site model. Injection rate is 2.78 μL/s
and data is acquired every second.

| | model parameters | | | | | | |
|---|---|---|---|---|---|---|---|
| | | | | | ΔH₁ | ΔH₂ | [L₂]_{T} |
| | ln K₁ | ln K₂ | n | m | (kJ mol⁻¹) | (kJ mol⁻¹) | (mM) |
| A | 6 | 4 | 1 | 1 | −30 | −10 | 4 |
| B | 7 | 4 | 1 | 1 | −30 | −10 | 4 |
| C | 8 | 4 | 1 | 1 | −30 | −10 | 4 |
| D | 9 | 4 | 1 | 1 | −30 | −10 | 4 |
| E | 7 | 4 | 0.5 | 1 | −30 | −10 | 4 |
| F | 7 | 4 | 2 | 1 | −30 | −10 | 4 |
| G | 7 | 4 | 1 | 0.5 | −30 | −10 | 4 |
| H | 7 | 4 | 1 | 2 | −30 | −10 | 4 |
| I | 7 | 4 | 1 | 1 | −20 | −10 | 4 |
| J | 7 | 4 | 1 | 1 | −40 | −10 | 4 |
| K | 7 | 4 | 1 | 1 | −30 | −10 | 2 |
| L | 7 | 4 | 1 | 1 | −30 | −10 | 8 |

| | | | | calculated thermodynamic | | | |
|---|---|---|---|---|---|---|---|
| | data at inflection point | | | parameters | | | |
| | [L₁]_{T} | VΔHₐₚₚ/2 | slope | | | | ΔH₁ |
| | (mM) | (J M⁻¹) | (kJ M⁻²) | ln K₁ | ln Kₐₚₚ | n | (kJ mol⁻¹) |
| A | 0.949 | −10.24 | −25.43 | 6.000 | 4.387 | 1.010 | −30.33 |
| B | 0.994 | −10.14 | −79.22 | 7.000 | 5.387 | 1.002 | −30.05 |
| C | 0.999 | −10.01 | −250.00 | 8.010 | 6.397 | 1.000 | −29.78 |
| D | 1.000 | −10.45 | −789.99 | 8.972 | 7.359 | 1.001 | −30.66 |
| E | 1.995 | −12.60 | −69.42 | 6.998 | 5.385 | 0.500 | −30.08 |
| F | 0.494 | −5.29 | −58.33 | 6.997 | 5.384 | 2.006 | −30.15 |
| G | 0.993 | −5.30 | −41.39 | 7.000 | 5.387 | 1.003 | −30.15 |
| H | 0.995 | −12.61 | −98.17 | 6.997 | 5.385 | 1.001 | −30.10 |
| I | 0.994 | −5.15 | −40.12 | 6.997 | 5.384 | 1.001 | −20.07 |
| J | 0.994 | −15.19 | −118.32 | 6.995 | 5.382 | 1.001 | −40.15 |
| K | 0.997 | −10.29 | −112.10 | 6.997 | 5.675 | 1.001 | −30.12 |
| L | 0.991 | −10.10 | −56.00 | 6.998 | 5.090 | 1.001 | −30.08 |

Approximation of concentration of [L₂] by [L₂]_{T} results in the observed difference between simulated and calculated parameters. However, the calculated ln K and n have less than 0.3% error, and values for ΔH₁ have less than 4% error for all simulations.

For the isotherm of two independent binding site model, the first and the third inflection points among three inflection points can be utilized to evaluate the six thermodynamic binding parameters as shown in FIG. 3C. The thermodynamic binding parameters from the simulation, data at inflection point of the simulated binding isotherm, and the calculated thermodynamic binding parameters by the algebraic equation for two independent binding sites model are summarized in Table 6 with a fixed molar enthalpy of binding for low affinity site of −10 kJ/mol.

TABLE 6
Evaluation of thermodynamic parameters at inflection point for
two independent binding sites model. Injection rate is 2.78 μL/s
and data is acquired every second.

| | model parameters | | | | | | |
|---|---|---|---|---|---|---|---|
| | [M₁]_{T} | | | ΔH₁ | [M₂]T | | |
| | (mM) | log K₁ | n | (kJ mol⁻¹) | (mM) | log K₂ | |
| A | 1 | 7 | 1 | −20 | 1 | 5 | 1 |
| B | 1 | 8 | 1 | −20 | 1 | 5 | 1 |
| C | 1 | 9 | 1 | −20 | 1 | 5 | 1 |
| D | 1 | 7 | 1 | −20 | 1 | 4 | 1 |
| E | 1 | 7 | 1 | −20 | 1 | 6 | 1 |
| F | 1 | 7 | 0.5 | −20 | 1 | 5 | 1 |
| G | 1 | 7 | 2 | −20 | 1 | 5 | 1 |
| H | 1 | | 1 | −20 | 1 | 5 | 0.5 |
| I | 1 | 7 | 1 | −20 | 1 | 5 | 2 |
| J | 1 | 7 | 1 | −15 | 1 | 5 | 1 |
| K | 1 | 7 | 1 | −30 | 1 | 5 | 1 |
| L | 0.5 | 7 | 1 | −20 | 1 | 5 | 1 |
| M | 2 | 7 | 1 | −20 | 1 | 5 | 1 |
| N | 1 | 7 | 1 | −20 | 0.5 | 5 | 1 |
| O | 1 | 7 | 1 | −20 | 2 | 5 | 1 |

| | data at inflection points | | | | | |
|---|---|---|---|---|---|---|
| | [L]_{T} | V(ΔH₁ + ΔH₂)/2 | slope | [L]_{T} | VΔH₂/2 | slope |
| | (mM) | (J M⁻¹) | (kJ M⁻²) | (mM) | (J M⁻¹) | (kJ M⁻²) |
| A | 1.000 | −14.94 | −24.99 | 1.990 | −4.86 | −25.25 |
| B | 1.000 | −14.95 | −79.47 | 1.989 | −5.00 | −25.03 |
| C | 1.000 | −14.95 | −251.20 | 1.990 | −5.00 | −25.00 |
| D | 1.000 | −14.51 | −82.95 | 1.898 | −5.03 | −7.94 |
| E | 1.001 | −14.99 | −7.14 | 1.998 | −5.54 | −82.95 |
| F | 2.001 | −15.09 | −17.73 | 2.989 | −5.15 | −25.48 |
| G | 0.495 | −14.94 | −35.28 | 1.490 | −5.05 | −25.13 |
| H | 0.990 | −14.97 | −17.57 | 2.989 | −5.04 | −17.75 |
| I | 1.006 | −14.83 | −35.87 | 1.488 | −5.20 | −36.25 |
| J | 1.001 | −12.43 | −12.68 | 1.989 | −5.05 | −25.06 |
| K | 1.000 | −19.93 | −49.63 | 1.989 | −5.15 | −25.62 |
| L | 0.495 | −14.94 | −35.28 | 1.489 | −5.05 | −25.13 |
| M | 2.011 | −14.91 | −17.73 | 2.989 | −5.15 | −25.48 |
| N | 1.006 | −14.82 | −35.87 | 1.488 | −5.18 | −36.25 |
| O | 0.990 | −14.95 | −17.57 | 2.989 | −5.04 | −17.75 |

| | calculated thermodynamic parameters | | | | | | |
|---|---|---|---|---|---|---|---|
| | | | | ΔH₁ | | | ΔH₂ |
| | log K₁ | log Kₐₚₚ | n | (kJ mol⁻¹) | log K₂ | m | (kJ mol⁻¹) |
| A | 7.003 | 4.967 | 0.989 | −20.16 | 5.033 | 1.001 | −9.72 |
| B | 8.019 | 6.015 | 0.999 | −19.89 | 5.001 | 1.001 | −10.00 |
| C | 9.018 | 7.014 | 1.000 | −19.89 | 5.000 | 1.000 | −10.00 |
| D | 7.182 | 6.143 | 0.999 | −18.96 | 3.998 | 1.002 | −10.06 |
| E | 7.106 | 4.155 | 0.934 | −18.90 | 5.952 | 1.002 | −11.08 |
| F | 7.036 | 5.042 | 0.497 | −19.88 | 4.990 | 1.002 | −10.30 |
| G | 7.034 | 5.027 | 1.983 | −19.78 | 5.001 | 0.996 | −10.09 |
| H | 7.021 | 4.717 | 0.991 | −19.86 | 4.999 | 0.498 | −10.08 |
| I | 7.107 | 5.424 | 0.990 | −19.25 | 4.981 | 2.029 | −10.40 |
| J | 7.074 | 5.077 | 0.991 | −14.76 | 4.993 | 1.001 | −10.10 |
| K | 7.029 | 5.031 | 0.991 | −29.55 | 4.995 | 1.001 | −10.31 |
| L | 7.034 | 5.029 | 0.991 | −19.78 | 4.999 | 0.996 | −10.09 |
| M | 7.062 | 5.077 | 0.994 | −19.52 | 4.986 | 1.012 | −10.30 |
| N | 7.103 | 5.417 | 0.990 | −19.28 | 4.984 | 1.015 | −10.36 |
| O | 7.024 | 4.721 | 0.991 | −19.82 | 4.999 | 0.995 | −10.08 |

Calculated thermodynamic binding parameters vary less than 3, 1, 6, 1, 1.5, and 12% from model parameters of ln K₁, n, ΔH₁, In K₂, in, and ΔH₂, respectively.

Experimental real-time evaluation of thermodynamic binding properties for single independent binding site, competitive binding site, and two independent binding sites model are summarized in Table 7.

TABLE 7
Evaluation of thermodynamic parameters with data
at inflection point(s) on experimental binding isotherms.

| | | | | Thermodynamic binding | | |
|---|---|---|---|---|---|---|
| | Data at inflection points | | | properties | | |
| | L_{inf} | VΔH/2 | slope | | | ΔH |
| Binding model | (mM) | (J M⁻¹) | (kJ M⁻²) | ln K | n | (kJ mol⁻¹) |
| Single independent | 0.991 | −10.576 | −205.54 | 14.22 | 1.008 | −21.15 |
| binding site^{a} | | | | | | |
| Competitive | 0.932 | −2.564 | −17.28 | 20.64 | 1.066 | −20.66 |
| binding site^{b} | | | | | | |
| Two independent | 0.429^{d} | −15.012^{d} | −211.75^{d} | 18.98^{f} | 1.047^{f} | −21.12^{f} |
| binding sites^{c} | 0.832^{e} | −4.451^{e} | −50.00^{e} | 12.24^{g} | 1.103^{g} | −8.90^{g} |

^{a}BaCl₂, 20 mM, in the 100 μL syringe was injected into 1 mM EDTA in a Tricine (20 mM) buffer solution in pH 8.5 at 25° C. Injection rate for the continuous injection mode is 1.7 μL/min.
^{b}Ca(NO₃)₂, 10 mM, in the 250 μL syringe was injected into the mixture of 1 mM EDTA and 4 mM BaCl₂ in a HEPES (20 mM) buffer solution in pH 8.5 at 25° C. The thermodynamic binding parameters for Ba²⁺ and EDTA were obtained independently and are 1.36 × 10⁶ M⁻¹, 1.00, and −14.57 kJ mol⁻¹ for K₂, m, and ΔH₂, respectively. Injection rate for the continuous injection mode is 10 μL/min.
^{c}Ca(NO₃)₂, 5 mM, in the 250 μL syringe was injected into the mixture of 0.45 mM EDTA and 0.45 mM DPTA in a Tricine (20 mM) buffer solution in pH 8.5 at 25° C. Injection rate for the continuous injection mode is 7.2 μL/min.
^{d}Data at the first inflection point
^{c}Data at the third inflection point
^{f}Thermodynamic binding properties of the association of Ca²⁺ and EDTA
^{g}Thermodynamic binding properties of the association of Ca²⁺ and DPTA

The thermodynamic binding parameters (In K, n, and ΔH) from the real-time evaluation differ from values using ITC equations by 1-10%. Although real-time method only uses data at the inflection point(s) of the binding isotherm, the estimated thermodynamic binding parameters are in good agreement with using the entire binding isotherm fit with an appropriate model.

### Example 2

### Materials

All materials were used without any further purification. Avidin, d-desthiobiotin, α-Lipoic acid, Ethylenediaminetetraacetic acid (EDTA) were obtained from Sigma-Aldrich.

Calcium nitrate tetrahydrate, and sodium hydroxide were obtained from Alfa Aesar. 4-(2-hydroxyethyl)-1-piperazineethanesulfonic acid (HEPES) was obtained from Research Organics, Inc.

Sample Preparations

Avidin, desthiobiotin, and α-Lipoic acid were prepared in deionized water. The molecular weight of avidin (12.8 units per mg protein) was used as 66000 g/mol. EDTA, and Ca(NO₃)₂ solutions were prepared with buffer solutions (HEPES). The acidity of these solutions was adjusted to pH 8.7 by NaOH solution (0.1 M). HEPES (1 mM) was used as the buffer solution.

Isothermal Titration Calorimetry

The continuous titrations were performed on a VP-ITC (Malvern Instruments, Westborough, Mass.) with a cell volume of 1.4 mL. The incremental titrations were performed on a NanoITC (TA Instruments, Lindon, Utah) with a cell volume of 1 mL. ITC experiments were carried out at 25° C. and the stirring speed was 270 rpm and 250 rpm for the continuous titration and incremental titration, respectively. For manipulating injection rate, a PHD 2000 syringe pump (Harvard Apparatus, Holliston, Mass.) was connected to the injection syringe of the VP-ITC.

Continuous Injection Mode for K_{d} in the Order of Nano- and Pico-Molar System

Cation chelation by EDTA derivatives is useful to test ITC model development. K_{d} of binding of Ca²⁺ and EDTA derivatives less than 10 nM have been determined indirectly through ITC. Determined K_{d} was influenced by buffer solution, pH, and titration methods. Binding of Ca²⁺ and EDTA was performed and analyzed with a single independent binding site model.

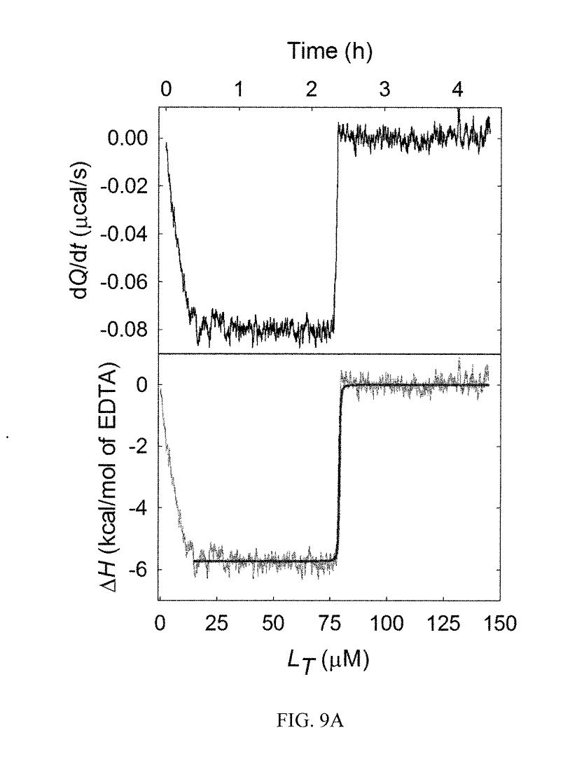

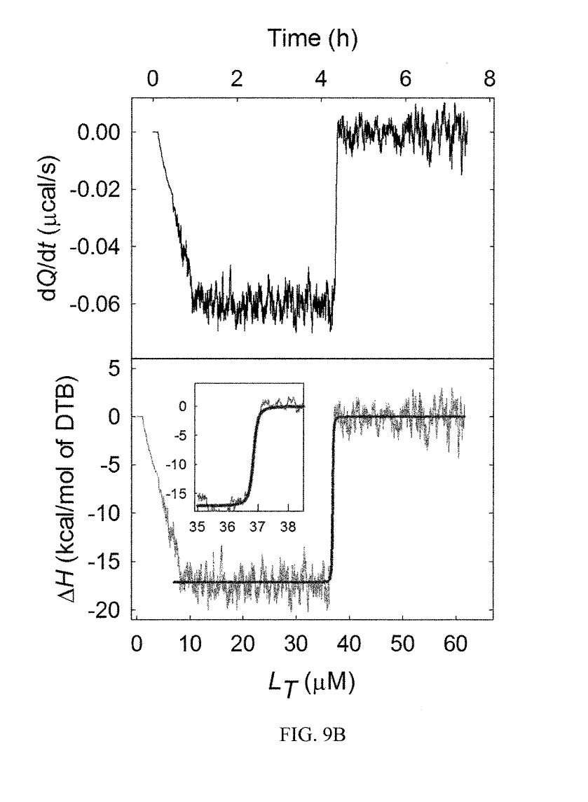

Single independent binding for high affinity binding Ca(NO₃)₂ (1 mM) in the syringe was injected into a 0.1 mM EDTA, see FIG. 9A, desthiobiotin (0.5 mM) in the syringe was injected into a 12.5 μM avidin, see FIG. 9B. Injection rate for Ca-EDTA binding system is 50 μL/h and the fit was done after 15 μM of ligand was injected. Injection rate for desthiobiotin-avidin binding system is 25 μL/h and the fit was done after 7 μM of ligand was injected.

FIG. 9A represents the raw data of binding isotherm (top) for binding of Ca²⁺ to EDTA at 25° C. through a continuous injection method and binding isotherm with respect to [L]_{T} and the corresponding best fit. The thermodynamic data summarized in Table 8.

TABLE 8
Thermodynamic binding parameters derived from continuous
injection method.

| | | | ΔH |
|---|---|---|---|
| Binding system | n | K | (kcal mol⁻¹) |
| Ca-EDTA^{a} | 0.932 | 0.87 nM | −5.73 |
| Avidin-desthiobiotin^{b} | 0.292 | 0.65 pM | −17.55 |

^{a}Ca(NO₃)₂ (10 mM) was injected with the flow rate of 50 μL/h into 1 mM EDTA in a HEPES (1 mM) buffer solution at pH. 8.7 at 25° C.
^{b}Desthiobiotin (0.5 mM) was injected with the flow rate of 25 μL/h into 12.5 μM avidin at 25° C. at pH 7.2.

The evaluated K_{d} for binding of Ca²⁺ and EDTA is in good agreement with the one evaluated with a competitive binding site model.

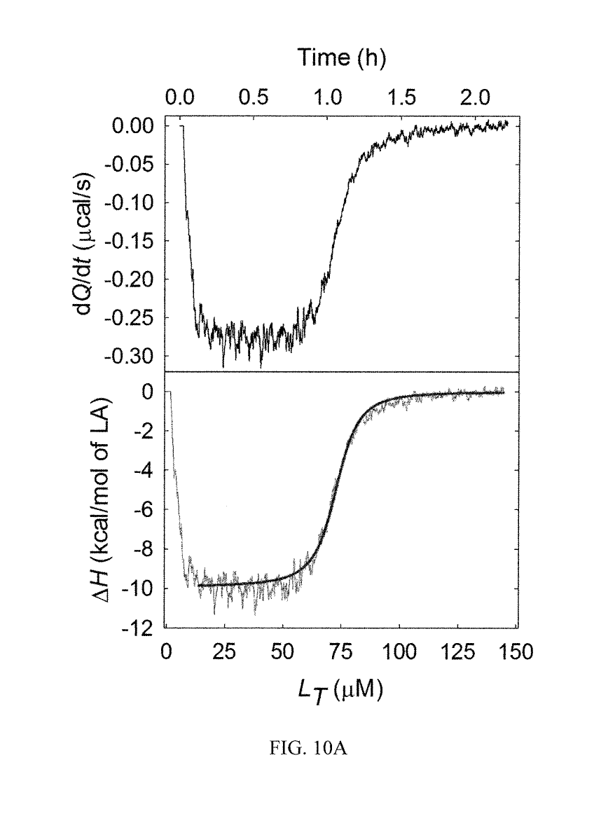

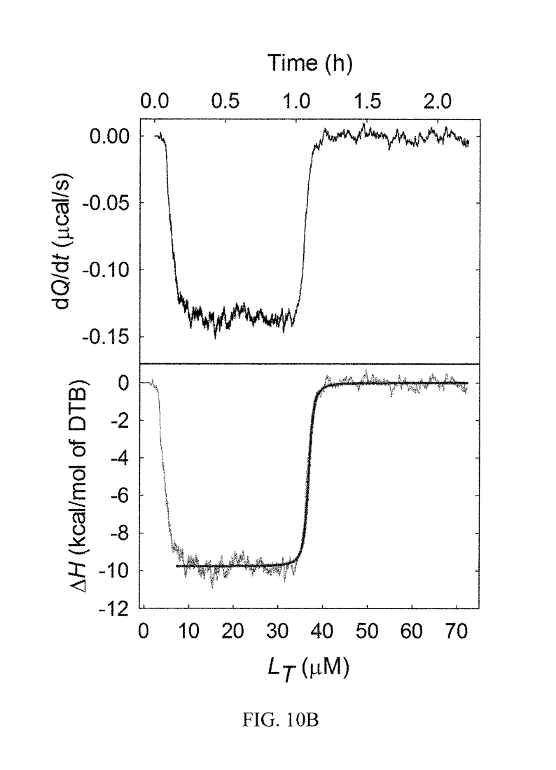

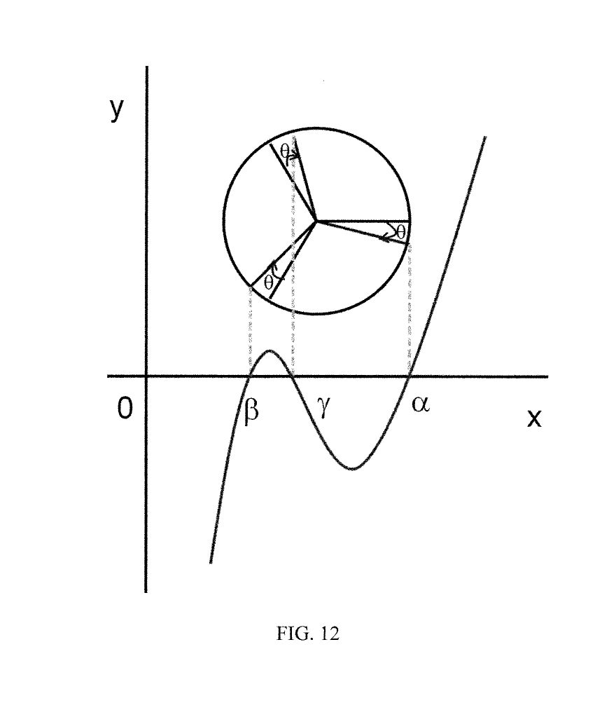

K_{d} of binding for avidin and desthiobiotin has been reported as 0.5 μM at pH 7 determined by UV-Vis spectroscopy. Binding of avidin and desthiobiotin was performed and analyzed with a single independent binding site model. FIG. 9B represents the raw data of binding isotherm (top) for binding of desthiobiotin to avidin at 25° C. through a continuous injection method and binding isotherm with respect to [L]_{T} and the corresponding best fit. The thermodynamic data summarized in Table 8. The measured pH for desthiobiotin-avidin system was 7.2. In order to obtain the thermodynamic binding properties of avidin-desthiobiotin, one can utilize the competitive binding site model with incremental injection method. A low-affinity ligand requires both K_{d} and K_{d,app}, apparent dissociation constant of binding high-affinity ligand and avidin bound with low-affinity ligand are less than 1 nM for feasible measurement with incremental injection mode. Lipoic acid, commercially available and active at similar pH of binding of avidin-desthiobiotin has a reported K_{d} of 0.7 μM at pH 6.8. The measured pH of the single independent binding site for lipoic acid-avidin system and competitive binding site for desthiobiotin-avidin mixed with lipoic acid system was 6.6. As seen in FIG. 12, the binding isotherm resembles a step function that shows less reliability for determining K_{d,app}. Therefore, competitive binding methods using incremental injection fail to measure the K_{d} of a ligand and receptor pair with a sub-picomolar K_{d}. K_{d} for avidin-lipoic acid and K_{d,app} are determined by continuous injection methods as shown in FIGS. 10A and 10B and the thermodynamic binding properties are summarized in Table 9.

TABLE 9
Thermodynamic binding parameters for determination of
desthiobiotin-avidin system via competitive binding model

| | |
|---|---|
| | Thermodynamic |
| Binding system | binding parameters |

| | | |
|---|---|---|
| Lipoic acid-avidin^{a} | n | 0.291 |
| | K_{d1} | 0.52 μM |
| | ΔH₁ (kcal mol⁻¹) | −9.95 |
| Desthiobiotin-avidin^{b} | m | 0.294 |
| (mixed with lipoic acid) | K_{d,app} | 1.04 nM |
| | ΔHₐₚₚ (kcal mol⁻¹) | −9.74 |
| Desthiobiotin-avidin^{c} | K_{d} | 0.72 pM |
| | ΔH (kcal mol⁻¹) | −19.7 |

^{a}Lipoic acid (1 mM) was injected with the flow rate of 0.1 mL/h into 25 μM avidin at 25° C. at pH 6.6.
^{b}Desthiobiotin (0.5 mM) was injected with the flow rate of 25 μL/h into 12.5 μM avidin mixed with 0.75 mM of lipoic acid at 25° C. at pH 6.6.
^{c}Thermodynamic binding parameters are evaluated by competitive binding method.

Table 9 shows that K_{d,app} is sub-nanomolar which is beyond the reliable measurement range of incremental titration method. Addition to determination of K_{d}, ΔH for binding of desthiobiotin and avidin determined by competitive binding site model has discrepancy from the direct measurement of ΔH by both incremental and continuous titration methods. This might be the consequence of different pH in the presence of lipoic acid during competitive binding experiment.

FIG. 10A shows results of single independent binding for lipoic acid (1 mM) in the syringe was injected into a 25 μM avidin solution. Injection rate for the continuous injection mode was 0.1 mL/h. The fit for the continuous injection method was done after 12 μM of ligand was injected. FIG. 10B shows competitive binding for desthiobiotin (0.5 mM) in the syringe was injected into a 12.5 μM avidin aqueous solution mixed with lipoic acid (0.75 mM). Injection rate for the continuous injection mode is 0.1 mL/h. The fit for the continuous injection method was done after 8 M of ligand was injected.

Injection Rate and Acquisition Criteria for a High Affinity Ligand and Receptor

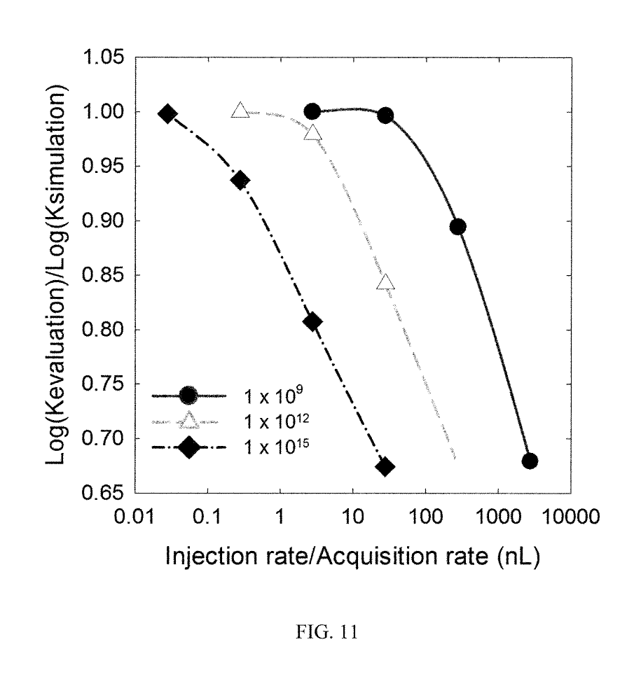

Due to how data is acquired, the finite time between data points during incremental injection causes error in the evaluation of the equilibrium constant. To obtain accurate thermodynamic parameters, the data acquired should be the real isotherm. Collecting a large number of data near the inflection point is important because the slope at the inflection point is a function of K. A large number of data points can be obtained by either slow injection rate or high acquisition rate. The injection rate and acquisition rate are inversely proportional to each other e.g. half of the injection rate with certain acquisition rate results in the same number of data point for a certain injection rate with double acquisition rate. FIG. 11 shows an example of the evaluation of binding constant for high affinity ligand and receptor (K_{d}=nano-molar, pico-molar, and femto-molar) depending on the injection rate and acquisition rate. The concentration of receptor in 1 mL reaction cell and ligand in the injection syringe is 1 mM and 20 mM, respectively. The enthalpy of binding is −20 kJ/mol and the binding stoichiometry is unity. This demonstrates that smaller injection rate/acquisition rate leads to more accurate determination of K. Either low injection rate or fast acquisition rate increases the number of data points near the inflection point. If a sufficient number of data are available around the inflection point, the slope can be used to determine the true K. Therefore, all parameters of the expression of the slope at the inflection point influence the number of data points at a given injection rate and acquisition rate. The expression at the inflection point is

$$\frac{{d}^{2} Q}{d {[ L ]}_{T}^{2}} {\vert}_{{[ L ]}_{T} = {[ L ]}_{T , inf}} = - \frac{V Δ H}{4} \sqrt{\frac{Kn}{{[ M ]}_{T}}} \qquad ( 36 )$$

The equation is changed in terms of time by multiplying (d[L]_{T}/dt)² for both sides because raw data (Q as function of t) from the ITC is a heat flow.

$$\frac{{d}^{2} Q}{d {t}^{2}} {\vert}_{{[ L ]}_{T} = {[ L ]}_{T , inf}} = - \frac{V Δ H}{4} \sqrt{\frac{Kn}{{[ M ]}_{T}}} {( \frac{d {[ L ]}_{T}}{d t} )}^{2} \qquad ( 37 )$$

Use of flow rate, v, and the concentration of ligand in the injection syringe, [L]_{S}, instead of d[L]_{T}/dt helps experiment design.

$$\frac{{d}^{2} Q}{d {t}^{2}} {\vert}_{{[ L ]}_{T} = {[ L ]}_{T , inf}} = - \frac{V Δ H}{4} \sqrt{\frac{Kn}{{[ M ]}_{T}}} {( \frac{{[ L ]}_{S} v}{V} )}^{2} \qquad ( 38 )$$

Finally, the slope of the binding isotherm in terms of time at the inflection point is

$$\frac{{d}^{2} Q}{d {t}^{2}} {\vert}_{{[ L ]}_{T} = {[ L ]}_{T , inf}} = - \frac{Δ H}{4} \sqrt{\frac{Kn}{{[ M ]}_{T}}} {( \frac{{[ L ]}_{S} v}{V} )}^{2} \qquad ( 39 )$$

The slope increases with an increase in ΔH, [L]_{S}, and v, and a decrease by [M]_{T}, and V. Therefore, reduction of [L]_{S} and v decreases the slope of the binding isotherm and increase the number of data points near the inflection point. An increase in [M]_{T} reduces the slope according to eqn. 39. However, large [M]_{T} requires large injection volume of L which leads to a loss of M in the cell due to the constant volume of the ITC. A change of [L]_{S} influences the slope of the binding isotherm by the second order of [L]_{S} while only square root of [M]_{T} increases the slope of the binding isotherm by change of [M]_{T} Therefore, reduction of both [M]_{T} and [L]_{S} results in the decrease of slope of the binding isotherm overall.

Details of the solution of a cubic equation, coefficients of the binding isotherm and the derivative of the coefficients, and the derivation of the algebraic equation at the inflection point are shown hereinbelow.

Solution of a Cubic Equation

The cubic equations for both competitive binding and two independent sites have positive discriminants. The cubic function is given by

f(x)=ax³+bx²+cx+d=0, (S1)

which has three real roots α, δ, and γ. The three real roots for a cubic equation, α, β, and γ, is shown in FIG. 12. The number of real roots can be determined by the discriminant, [symbol] , for cubic equation (eqn. S1) is given by

[symbol]

=18abcd−4b³d+b²c²−4ac³−27a²d², (S2)

Note that [symbol] >0 for both competitive binding and two individual binding site models.

By substituting x=z−b/(3a), eqn. S1 has the form

az³−3aδ²z+q=0, (S3)

so that

$$\begin{matrix}q = \frac{2 {b}^{3} - 9 abc + 27 {a}^{2} d}{27 {a}^{2}} , \\ and & ( S4 ) \\ {δ}^{2} = \frac{{b}^{2} - 3 ac}{9 {a}^{2}} . & ( S5 )\end{matrix}$$

By using z=2δ cos θ, eqn. S3 becomes

h(4 cos³θ−3 cos θ)+q=0, (S6)

where h=2aδ³, and eqn. S6 gives

θ=arccos(−q/h)/3. (S7)

The three real roots for eqn. S1 are

$$\{ \begin{matrix}α = - \frac{b}{3 a} + 2 δ cos θ \\ β = - \frac{b}{3 a} + 2 δ cos ( \frac{2 π}{3} + θ ) \\ γ = - \frac{b}{3 a} + 2 δ cos ( \frac{4 π}{3} + θ )\end{matrix} \qquad ( S8 )$$

and their derivatives with respect to the injected ligand are

$$\{ \begin{matrix}{α}^{′} = - \frac{{b}^{′}}{3 a} + 2 {δ}^{′} cos θ - 2 δ {θ}^{′} sin θ \\ {β}^{′} = - \frac{{b}^{′}}{3 a} + 2 {δ}^{′} cos ( \frac{2 π}{3} + θ ) - 2 δ {θ}^{′} sin ( \frac{2 π}{3} + θ ) \\ {γ}^{′} = - \frac{{b}^{′}}{3 a} + 2 {δ }^{′} cos ( \frac{4 π}{3} + θ ) - 2 δ {θ}^{′} sin ( \frac{4 π}{3} + θ )\end{matrix} \qquad ( S9 )$$

The derivatives for parameters with respect to the injected ligand for eqn. S9 is given by

$$\begin{matrix}{δ}^{′} = \frac{2 {bb}^{′} - 3 {ac}^{′}}{6 {a}^{2} \sqrt{( {b}^{2} - 3 ac ) \text{/} {a}^{2}}} , & ( S 10 ) \\ {q}^{′} = \frac{2 {b}^{2} {b}^{′} - 3 {ab}^{′} c - 3 {abc}^{′} + 9 {a}^{2} {d}^{′}}{9 {a}^{2}} , & ( S 11 ) \\ {h}^{′} = 6 a {δ}^{2} {δ}^{′} , \\ and & ( S 12 ) \\ {θ}^{′} = \frac{{hq}^{′} - {h}^{′} q}{3 {h}^{2} \sqrt{1 - {q}^{2} \text{/} {h}^{2}}} . & ( S 13 )\end{matrix}$$

Coefficients of the ITC Equation for Binding Isotherms

The ITC equation for the binding isotherms of the competitive binding site and the two independent binding sites model require solving cubic equations of heat as a function of the total concentration of ligand and derivative of heat in terms of the total concentration of ligand. The coefficients for eqns. (11), (12), (16) and (17), and the derivatives of the coefficients in terms of the total concentration of ligands are shown below.

Coefficients of the equation for competitive binding site model

A=K₁²mn−K₁K₂n²

B=−K₁²m[M]_{T}−K₁mn−2K₁²[L₁]_{T}mn−K₁K₂[L₂]_{T}mn+K₁K₂[M]_{T}n+K₂n²+K₁K₂[L₁]_{T}n²

C=2K₁²[L₁]_{T}m[M]_{T}+K₁[L₁]_{T}mn+K₁²[L₁]_{T}²mn+K₁K₂[L₁]_{T}[L₂]_{T}mn−K₁K₂[L₁]_{T}[M]_{T}n

D=−K₁²[L₁]_{T}m[M]_{T}

E=−K₁K₂m²+K₂²mn

F=K₁m²+K₁K₂[L₂]_{T}m²+K₁K₂m[M]_{T}−K₂mn−K₁K₂[L₁]_{T}mn−2K₂²[L₂]_{T}mn−K₂²[M]_{T}n

G=−K₁K₂[L₂]_{T}m[M]_{T}+K₂[L₂]_{T}mn+K₁K₂[L₁]_{T}[L₂]_{T}mn+K₂²[L₂]_{T}²mn+2K₂²[L₂]_{T}[M]_{T}n

H=−K₂²[L₂]_{T}²[M]_{T}n (S14)

A′=dA/d[L₁]_{T}=0

B′=dB/d[L₁]_{T}=−2K₁²mn+K₁K₂n²

C′=dC/d[L₁]_{T}=2K₁²m[M]_{T}+K₁mn+2K₁²[L₁]_{T}mn+K₁K₂[L₂]_{T}mn−K₁K₂[M]_{T}n

D′=dD/d[L₁]_{T}=−2K₁²[L₁]_{T}m[M]_{T}

E′=dE/d[L₁]_{T}=0

F′=dF/d[L₁]_{T}=−K₁K₂mn

G′=dG/d[L₁]_{T}=K₁K₂[L₂]_{T}mn

H′=dH/d[L₁]_{T}=0 (S15)

Coefficients of the equation for two independent binding sites model

A=K₁²mn²−K₁K₂mn²

B=−2K₁²m[M₁]_{T}n+K₁K₂m[M₁]_{T}n−K₁mn²+K₁mn²−K₁²[L]_{T}mn²+K₁K₂[L]_{T}mn²−K₁K₂[M₂]_{T}n²

C=K₁²m[M₁]_{T}²+K₁m[M₁]_{T}n+2K₁²[L]_{T}m[M₁]_{T}n−K₁K₂[L]_{T}m[M₁]_{T}n+K₁K₂[M₁]_{T}[M₂]_{T}n

D=−K₁²[L]_{T}m[M₁]_{T}²

E=−K₁K₂m²n+K₂²m²n

F=−K₁K₂m²[M₁]_{T}+K₁m²n−K₂m²n+K₁K₂[L]_{T}m²n−K₂²[L]_{T}m²n+K₁K₂m[M₂]_{T}n−2K₂²m[M₂]_{T}n

G=K₁K₂m²[M₁]_{T}[M₂]_{T}+K₂m[M₂]_{T}n−K₁K₂[L]_{T}m[M]_{T}n+2K₂²[L]_{T}m[M₂]_{T}n+K₂²[M₂]_{T}n

H=−K₂²[L]_{T}[M₂]_{T}²n (S16)

A′=dA/d[L]_{T}=0

B′=dB/d[L]_{T}=−K₁²mn²+K₁K₂mn²

C′=dC/d[L]_{T}=2K₁²m[M₁]_{T}n−K₁K₂m[M₁]_{T}n

D′=dD/d[L]_{T}=−K₁²m[M₁]_{T}²

E′=dE/d[L]_{T}=0

F′=dF/d[L]_{T}=K₁K₂m₂n−K₂²m²n

G′=dG/d[L]_{T}=−K₁K₂m[M₂]_{T}n+2K₂²m[M₂]_{T}n

H′=dH/d[L]_{T}=−K₂²[M₂]²n (S17)

P Derivation of the values at an inflection point

From the binding isotherm as a function of [L]_{T} (eqn. 7), the first derivative of the binding isotherm is given by

$$\frac{{d}^{2} Q}{d {[ L ]}_{T}^{2}} = - V Δ H \frac{{d}^{2} [ SL ]}{d {[ L ]}_{T}^{2}} = - V Δ H \frac{2 {K}^{2} {{n}^{2} [ M ]}_{T}}{{( {( {K [ M ]}_{T} + n + {K [ L ]}_{T} n )}^{2} - 4 {{{K}^{2} [ L ]}_{T} [ M ]}_{T} n )}^{3 \text{/} 2}} \qquad ( S 18 )$$

The second derivative of the binding isotherm is

$$\frac{{d}^{3} Q}{d {[ L ]}_{T}^{3}} = - V Δ H \frac{{d}^{3} [ SL ]}{d {[ L ]}_{T}^{3}} = V Δ H \frac{6 {K}^{3} {{n}^{3} [ M ]}_{T} ( - {K [ M ]}_{T} + n + {K [ L ]}_{T} n )}{{( {( {K [ M ]}_{T} + n + {K [ L ]}_{T} n )}^{2} - 4 {{{K}^{2} [ L ]}_{T} [ M ]}_{T} n )}^{5 \text{/} 2}} \qquad ( S 19 )$$

The x-axis at the inflection point where

$$\frac{{d}^{3} Q}{d {[ L ]}_{T}^{3}} = 0 \qquad ( S20 )$$

yields

$${[ L ]}_{T , inf} = \frac{{[ M ]}_{T}}{n} - \frac{1}{K} \qquad ( S21 )$$

By substituting [L]_{T} in eqn. 7 with [L]_{T,inf}, the y-axis at the inflection point becomes

$$\frac{d Q}{d {[ L ]}_{T}} {\vert}_{{[ L ]}_{T} = {[ L ]}_{T , inf}} = - \frac{V Δ H}{2} \qquad ( S 22 )$$

By substituting [L]_{T} in eqn. S18 with [L]_{T,inf}, the slope at the inflection point becomes

$$\frac{{d}^{2} Q}{d {[ L ]}_{T}^{2}} {\vert}_{{[ L ]}_{T} = {[ L ]}_{T , inf}} = - \frac{V Δ H}{4} \sqrt{\frac{Kn}{{[ M ]}_{T}}} \qquad ( S23 )$$

Any patents or publications mentioned in this specification are incorporated herein by reference to the same extent as if each individual publication is specifically and individually indicated to be incorporated by reference.

The methods described herein are presently representative of preferred embodiments, exemplary, and not intended as limitations on the scope of the invention. Changes therein and other uses will occur to those skilled in the art. Such changes and other uses can be made without departing from the scope of the invention as set forth in the claims.

1. An isothermal titration calorimetry method for determining one or more binding characteristics of a ligand and a receptor, comprising:
injecting a ligand into a sample cell of a calorimeter, the sample cell containing a receptor, wherein the injecting is continuous;
obtaining heat flow values indicative of binding of the ligand to the receptor; and
calculating the one or more binding characteristics in real-time, producing one or more determined binding characteristics.

2. The isothermal titration calorimetry method of claim 1, wherein calculating the one or more binding characteristics comprises calculation of the total concentration of ligand injected.

3. The isothermal titration calorimetry method of claim 1, wherein the ligand and receptor are characterized by high binding affinity where K_{d} is lower than 1 nM.

4. The isothermal titration calorimetry method of claim 1, wherein no additional heat flow values are obtained once the second derivative is determined to be equal to zero, thereby shortening the time to producing one or more determined binding characteristics compared to an incremental isothermal titration calorimetry method.

5. The isothermal titration calorimetry method of claim 1, wherein a syringe pump is used for continuously injecting the ligand.

6. A computer program for determining one or more binding characteristics of a ligand and a receptor using heat flow values obtained by an isothermal titration calorimetry method, the computer program operative to calculate the one or more binding characteristics in real-time, producing one or more determined binding characteristics displayed to a user.

7. The computer program of claim 6, operative to calculate the one or more binding characteristics by incorporating calculation of the total concentration of ligand injected into a sample cell of a calorimeter.

8. The computer program of claim 6, wherein the ligand and receptor are characterized by high binding affinity where K_{d} is lower than 1 nM.

9. The computer program of claim 6, wherein the program provides a signal indicating that no additional heat flow values are obtained once the second derivative is determined to be equal to zero.

10. An isothermal titration calorimeter in signal communication with a computer, the computer having a program according to claim 6.

11. The isothermal titration calorimeter of claim 10, comprising a syringe pump for continuous injection of a ligand into the sample cell of the isothermal titration calorimeter.

12. An isothermal titration calorimetry method for determining one or more binding characteristics of a ligand and a receptor, comprising:
injecting a ligand into a sample cell of a calorimeter, the sample cell containing a receptor, wherein the injecting is continuous;
obtaining heat flow values indicative of binding of the ligand to the receptor; and
calculating the binding characteristic, wherein calculating the one or more binding characteristics comprises calculation of the total concentration of ligand injected, producing one or more determined binding characteristics.

13. The isothermal titration calorimetry method of claim 12, wherein the calculating is in real-time.

14. The isothermal titration calorimetry method of claim 12, wherein the ligand and receptor are characterized by high binding affinity where K_{d} is lower than 1 nM.

15. The isothermal titration calorimetry method of claim 12, wherein no additional heat flow values are obtained once the second derivative is determined to be equal to zero, thereby shortening the time to producing one or more determined binding characteristics compared to an incremental isothermal titration calorimetry method.

16. The isothermal titration calorimetry method of claim 12, wherein a syringe pump is used for continuously injecting the ligand.

17. An isothermal titration calorimetry method for determining one or more binding characteristics of a ligand and a receptor, comprising:
injecting a ligand into a sample cell of a calorimeter, the sample cell containing a receptor, wherein the injecting is continuous;
obtaining heat flow values indicative of binding of the ligand to the receptor; and
calculating the one or more binding characteristics, wherein no additional heat flow values are obtained once the second derivative is determined to be equal to zero, thereby shortening the time to producing one or more determined binding characteristics compared to an incremental isothermal titration calorimetry method.

18. The isothermal titration calorimetry method of claim 17, wherein calculating the calculating is in real-time.

19. The isothermal titration calorimetry method of claim 17, wherein the ligand and receptor are characterized by high binding affinity where K_{d} is lower than 1 nM.

20. The isothermal titration calorimetry method of claim 17, wherein calculating the one or more binding characteristics comprises calculation of the total concentration of ligand injected, producing one or more determined binding characteristics.
